(19) United States
(12) Patent Application Publication
(10) Pub. No.: US 2023/0200688 A1
(43) Pub. Date: Jun. 29, 2023

(54) METABOLIC MONITORING SYSTEM

(71) Applicant: Zense-Life Inc., Carlsbad, CA (US)

(72) Inventors: Mitchell Steven Roslin, Amonk, NY (US); Robert James Boock, Carlsbad, CA (US); Eli Reihman, San Diego, CA (US); Leif Bowman, San Diego, CA (US); Todd Noboru Haseyama, Encinitas, CA (US); Katherine Yerre Koehler, Solana Beach, CA (US)

(73) Assignee: Zense-Life Inc., Carlsbad, CA (US)

(21) Appl. No.: 18/173,685

(22) Filed: Feb. 23, 2023

Related U.S. Application Data
(63) Continuation-in-part of application No. 16/386,021, filed on Apr. 16, 2019, now abandoned.
(60) Provisional application No. 62/659,537, filed on Apr. 18, 2018.

(51) Int. Cl. A61B 5/145; A61B 5/00

(52) U.S. Cl. CPC A61B 5/14532; A61B 5/7275; A61B 5/4866; A61M 2205/50

(57) ABSTRACT

A method for metabolic monitoring includes a processor receiving data associated with an individual from a metabolic sensor, the data comprising at least two of glucose data, ketone data and lactate data. The processor receives food intake information and physical activity information associated with the individual. The processor calculates a global metric and determines an individualized metric by correlating the food intake and the physical activity information to the global metric. The processor recommends a behavior modification based on the individualized metric.

### RELATED APPLICATIONS

This application is a continuation-in-part of U.S. patent application Ser. No. 16/386,021, filed on Apr. 16, 2019, and entitled “Metabolic Monitoring System”; which claims priority to U.S. Provisional Patent Application No. 62/659,537, filed on Apr. 18, 2018, and entitled “Metabolic Monitoring System”; all of which are hereby incorporated by reference in full.

### BACKGROUND

Monitoring glucose levels is critical for diabetes patients. Continuous glucose monitoring (CGM) sensors are a type of device in which fluid is sampled from just under the skin multiple times a day. CGM devices typically involve a small housing in which the electronics are located and which is adhered to the patient's skin to be worn for a period of time. A CGM sensor, which is often electrochemical, is delivered subcutaneously by a small needle within the device.

Electrochemical glucose sensors operate by using electrodes which detect an amperometric signal caused by oxidation of enzymes during conversion of glucose to gluconolactone. The amperometric signal can then be correlated to a glucose concentration. Two-electrode (also referred to as two-pole) designs use a working electrode and a reference electrode, where the reference electrode provides a reference against which the working electrode is compared. Three-electrode (or three-pole) designs have a working electrode, a reference electrode and a counter electrode. The counter electrode replenishes ionic loss at the reference electrode and is part of the ionic circuit.

Glucose readings taken by the sensor can be tracked and analyzed by a monitoring device, such as by scanning the sensor with a customized receiver or by transmitting signals to a smartphone or other device that has a specific software application. Software features that have been included in CGM systems include viewing glucose levels over time, indicating glucose trends, and alerting the patient of high and low glucose levels.

### SUMMARY

In some embodiments, a method includes receiving, by a processor, data associated with an individual from a metabolic sensor, the data comprising at least two of glucose data, ketone data and lactate data; and receiving, by the processor, food intake information and physical activity information associated with the individual. The processor calculates a global metric that is based on i) the glucose data and the ketone data, ii) the glucose data and the lactate data, iii) the ketone data and the lactate data; or iv) the glucose data, the ketone data and the lactate data. The processor determines an individualized metric by correlating the food intake information and the physical activity information to the global metric and recommends a behavior modification based on the individualized metric.

In some embodiments, a method includes receiving, by a processor, data associated with an individual from a metabolic sensor, the data comprising at least two of glucose data, ketone data, and lactate data; and receiving, by the processor, food intake information and physical activity information associated with the individual. The processor calculates a global metric, the global metric being an indicator ratio comprising at least two of the glucose data, the ketone data, and the lactate data. The processor determines an individualized metric by correlating the food intake information and the physical activity information to the global metric and recommends a behavior modification based on the individualized metric.

### BRIEF DESCRIPTION OF THE DRAWINGS

FIG. 1 shows a graph of post-prandial glucose response for various individuals in a prior art study.

FIG. 2 is a comparison of glucose variability values calculated using various methods.

FIGS. 3A-3B and FIG. 4 are graphs of glucose parameters over time.

FIG. 5A is a schematic of a metabolic monitoring system, in accordance with some embodiments.

FIGS. 5B-5E show schematics of metabolic monitoring devices, in accordance with some embodiments.

FIG. 5F is a schematic of the server of FIG. 5A, in accordance with some embodiments.

FIGS. 6A-6C show flowcharts of methods for monitoring metabolic activity, in accordance with some embodiments.

FIG. 7 is a tree diagram showing inputs that can be used in the present methods, in accordance with some embodiments.

FIGS. 8 and 9 show examples of user interfaces, in accordance with some embodiments.

FIGS. 10A and 10B are embodiments of a user interface for the display of data, in accordance with some embodiments.

FIGS. 11A-11C illustrate user interfaces for the display of data, in accordance with some embodiments.

FIGS. 12A-12C depict embodiments of a user interface to communicate data, in accordance with some embodiments.

FIGS. 13A-13C depict other embodiments of a user interface to communicate data, in accordance with some embodiments.

FIG. 14A shows an embodiment of a secondary user interface display screen to display data, in accordance with some embodiments.

FIG. 14B shows examples of simple visuals of suggested foods, drinks or medications, in accordance with some embodiments.

FIG. 15A shows the user interface with a dropdown menu to further communicate data, in accordance with some embodiments.

FIG. 15B is a secondary user interface display screen to display data, in accordance with some embodiments.

FIG. 16A is a user interface for the display of data, in accordance with some embodiments.

FIGS. 16B and 16C depict embodiments of example software applications in communication with the system.

FIG. 17 is a user interface for the display of data, in accordance with some embodiments.

FIG. 18 is a user interface for the display of data, in accordance with some embodiments.

FIGS. 19A-19C are flowcharts of methods for determining metrics for an individual, in accordance with some embodiments.

FIGS. 20A and 20B are example graphs of utilizing an individual metric, in accordance with some embodiments.

### DETAILED DESCRIPTION

Embodiments of the present disclosure uniquely use direct real-time metabolic data to encourage a user to change or modify behavior related to, for example, eating, exercise and subsequent weight loss. Methods and systems are disclosed in which continuous glucose monitoring is used to provide real-time feedback on the impact of eating various foods on post-prandial (post-meal) glucose levels in an individual. A goal of embodiments of the present methods and systems is to encourage patients to lower the amplitude and number of glucose spikes following eating. While individuals eat and drink according to their likes and dislikes and until a feeling of satiety, the impact of the food and drinks on their body chemistry is unknown. The higher the glucose spike the higher the production of insulin, which leads to fat formation, a crescendo and decrescendo in glucose values, and a greater likelihood of eating more frequently. Each individual reacts to different foods in a unique manner. In embodiments herein, providing information that correlates food intake to glucose metrics can dramatically alter food choices and improve health. Furthermore, weight, exercise, and stress can change these reactions, requiring frequent recalibration. Embodiments can account for the fact that the body is always changing.

Methods and systems are also disclosed in which a device monitors glucose, ketone, and/or lactate to provide real-time feedback on food intake and physical activity (e.g., exercise, workouts), to help individuals adjust or optimize their habits. For example, methods of the present disclosure can analyze data on glucose, ketone, and/or lactate measured from a user to make recommendations on how the user can modify their behavior to work toward their weight loss or fitness goals. Recommendations can include food parameters (e.g., what types, amounts, and order of food to eat) and/or exercise parameters (e.g., when or how to exercise) to achieve health benefits, such as desired aerobic and fat burning responses. The methods can provide analyses that are customized to the individual, based on how their body processes food and reacts to physical activity.

Monitoring of the metabolic substances in this disclosure may be performed by sampling blood, interstitial fluid, or a combination of these. “Continuous” monitoring may be considered in this disclosure as being on an ongoing basis over time, such as taking measurements multiple times a day. Depending upon the patient's specific medical needs, the continuous monitoring may be performed at different intervals. For example, some continuous metabolic monitors may be set to take multiple readings per minute, whereas in other cases the continuous metabolic monitor can be set to take readings every hour or so. It will be understood that a continuous metabolic monitor may sense and report metabolic readings at different intervals, and the reading rate may change depending on past measurements, time of day, or other criteria.

Embodiments disclose sensor-based systems for weight loss, treatment of insulin resistance and its related diseases such as polycystic ovary syndrome (PCOS), non-alcoholic steatohepatitis (NASH), non-alcoholic fatty liver disease (NAFLD), and reducing the likelihood of cancer recurrence. These diseases are related to excess glucose in the body. The present systems may include a monitoring system with feedback and professional advice via calculated metrics to help treat the person's obesity or weight management regime, and then creating actionable goals that are communicated on a software application for the patient to utilize and modify their behavior. Embodiments can also be used for general health monitoring and improvement.

Although some embodiments shall be described in terms of providing weight loss recommendations for a user, the concepts can be applied to providing other user-derived behavior modifications or correlations. For example, the present methods and systems can recommend behavior modifications such as training programs for athletes, or health management recommendations in relation to a medical condition of a user.

The system includes a software application and a sensor/transceiver that wirelessly communicates to a device which can be, for example, a smartphone, tablet, smart watch, or the like. In some embodiments, all the information is sent to a cloud-based server for analysis. In other embodiments, the information and processing of the data can be performed on the device or by an electronics unit connected to the metabolic sensing device. In further embodiments, the sensor/transceiver data can be sent to other types of devices, such as a desktop computer or a kiosk that may be located in a facility such as a doctor's office or hospital. Analytics, which can be cloud-based or can be included in the software application of the device, create personalized advice with personalized metrics based directly on the individual's data and can be distributed back to the patient and/or sent to a physician, dietician, trainer or family member.

In some embodiments, the metrics are based upon glucose variability and total glucose load, uniquely combining multiple glucose indicators to form a global metric that serves as a metabolic index. The glucose information is sent to artificial intelligence (AI) programs, which may be cloud-based, to correlate the global metrics with food intake. The metrics and food intake information may also be correlated with numerous other measures such as heart rate (HR), location, activity, etc., to provide a more complete picture of the person's individual metabolic profile.

Many embodiments are included for displaying data on a user interface device. The data may be information such as the mean glucose level or another metric that is calculated or obtained from the sensor of the continuous glucose monitoring. The recommended behavior modifications may also be displayed which includes eating a food, drinking a beverage, taking medication, or performing an exercise. These are specific activities and quantities based on the historical food intake information received and historical metabolic index calculations for the specific individual. In some embodiments, the processor is part of a device such as a mobile phone having a lock screen, home screen or wallpaper feature. The lock screen, home screen or wallpaper may be modified based on the glucose data associated with an individual from a metabolic sensor. In other words, the lock screen, home screen and/or wallpaper of the device may be modified based on the glucose data or other metabolic data from the sensor(s).

Clinical evidence data from various trials and studies show a correlation between weight loss and glucose variation, a link between obesity and glucose variation, and a correlation between food intake and glucose variation.

Evidence of a correlation between weight loss and glucose variation was first shown in the FLAT-SUGAR trial. This trial compared insulin to GLP-1 Agonist (a drug that controls glucose variation) in the hope of improving A1c levels (a measure of glycosolated hemoglobin linked to long-term health risks). The study showed no marked change in A1c even when marked reductions in glucose variations were shown. However, an unexpected observation from the study was a dramatic and sustained weight loss (4.5 kg or 10.6 lbs over 26 weeks) in the group with dramatically reduced glucose variation.

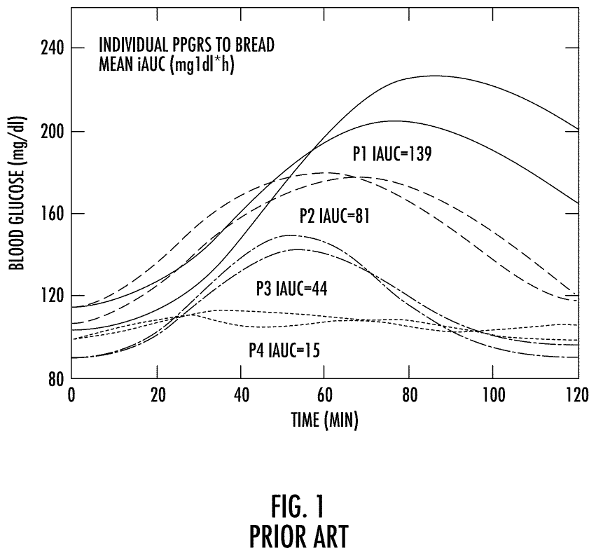

Evidence of a link between obesity and glucose variation was demonstrated in a paper by Salkind et al. This study showed that higher glucose variability exists in overweight, pre-diabetic and obese patients compared to non-diabetic adult controls. It remains to be demonstrated whether this is a cause or an effect. A study by Trico et al. showed that by simple changes in the order of food intake, glucose response can be altered. In other words, glucose variation can be reduced through changing the food intake sequence. This study, which focused on post-prandial (meal) peaks in glucose, demonstrates that simple advice can be used to modify glucose response to foods. A further consideration in correlating food intake to glucose response is that every individual responds to food differently. For example, FIG. 1 is a graph of post-prandial glucose response (PPGR) of the blood glucose in mg/dl over time showing the insulin area under the curve (IAUC). The graph shows that four people (P1, P2, P3, P4) given the exact same bread to eat had wide variations in their glucose response. For example, person 1 (P1) had an IAUC of 139 while person 4 (P4) had an IAUC of 15. It can be seen from FIG. 1 that weight loss products must be tailored to the individual and their physiologic response to be most effective as opposed to a one-size-fits-all approach.

In embodiments, systems and methods personalize a program to control blood glucose variations and overall glucose load for a consumer by using global metrics that are a unique combination of multiple glucose indicators. The global metrics are essentially metabolic indices and are utilized to determine an individualized metric, where the individualized metric is customized for that particular consumer. By controlling blood glucose levels, the consumer can improve their health which is beneficial for managing diseases such as diabetes and for weight loss.

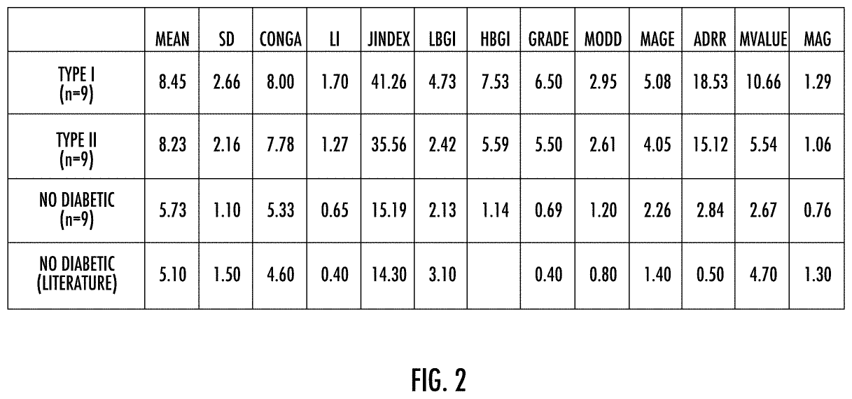

To derive the unique global metrics involving glucose data in the present disclosure, a pre-existing data set was first examined to calculate concepts and to determine whether continuous calculation of variation would provide new data or insights to use in a weight loss product. FIG. 2 shows a comparison of glucose variation across normal, Type I diabetic and Type II diabetics (normally associated with being overweight) calculated by a number of methods. The glucose variation (GV) data shown in FIG. 2 were 7-day averages calculated using EasyGV online software. The calculation types include mean, standard deviation (SD), continuous overall net glycemic action (CONGA), lability index (LI), J-index (J=0.324*(MBG+SD)²), low blood glucose index (LBGI), high blood glucose index (HBGI), glycemic risk assessment diabetes equation (GRADE), mean of daily differences (MODD), mean amplitude of glycemic excursions (MAGE), average daily risk ratio (ADRR), M-value of Schlichtkrull, and mean absolute glucose (MAG). The 7-day global calculations of these populations were in line with those reported in the literature, showing this data set to be representative. The calculations by the various GV methods show that a significant difference between diabetic and non-diabetic populations was observed.

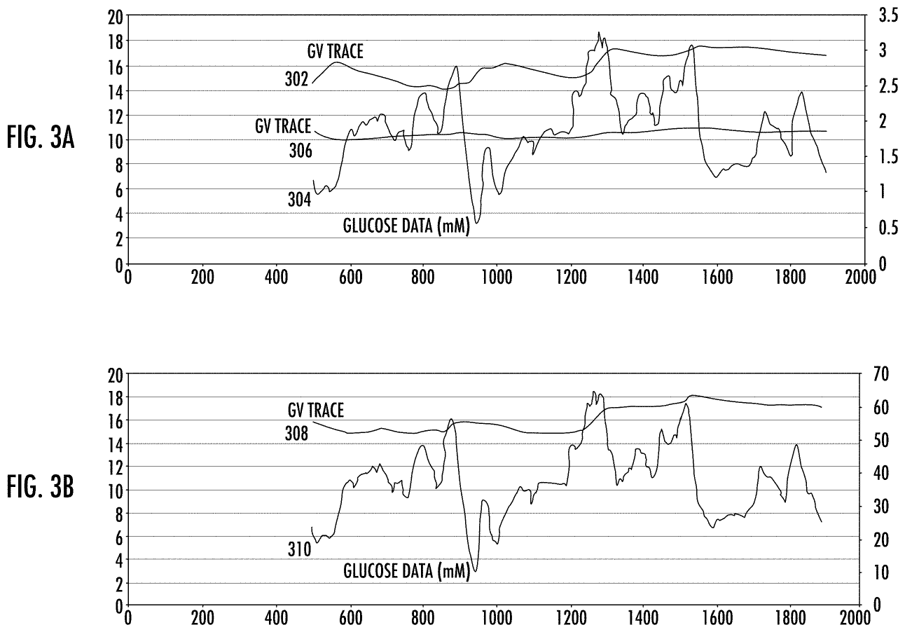

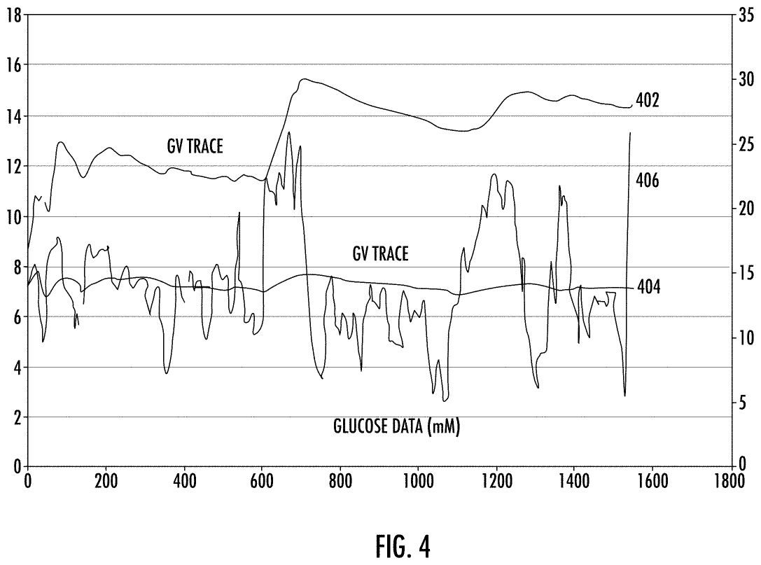

Next, a new concept of tracking glucose variability in real-time was investigated. FIGS. 3A-3B show continuous glucose variation calculations overlaying continuous glucose data for a sample Type I patient. FIG. 3A shows real-time glucose variation values calculated by SD (GV Trace 302), compared to glucose data values, line 304. FIG. 3A also shows the average glucose or Glucose Load, labeled as the mean (line 306). FIG. 3B is similar to FIG. 3A but uses the J-index method for the GV Trace, line 308 compared to glucose data values, line 310. FIG. 4 is a graph for another Type I patient, showing a J-index GV Trace as line 402, a mean GV trace as line 404, and glucose data values as line 406. These graphs demonstrate that continuous calculation of glucose variation is distinct from continuous glucose values and thus offers more and different data to be used in making a weight loss product. For example, the slopes, locations of peaks and valleys, and trends for glucose variability are different from those for the glucose values. As a specific example, the glucose variation (J-index line 402) in FIG. 4 continues to climb over the duration, indicating the blood glucose variations and overall glucose load of the person is out of control.

Embodiments uniquely use the concept of monitoring real-time glucose variation to formulate metrics for a weight loss program, where the metrics are personalized for an individual's specific characteristics. Terminology used in calculating glucose metrics is listed below:
GV=glucose variability (calculated by any number of formulas, such as those known in the art);
GL=glucose load or the average glucose value over a time period, such as 1 day;
PPP=post-prandial (meal) peak;
Global Metric=metric involving more than one type of metabolic indicator.

The global metrics are unique indices of the present embodiments that use weighting factors to combine metabolic indicators. In some examples, a global metric may be for glucose, combining the metabolic indicators GV, GL and PPP. The weighting factors are tailored for the data of an individual person, such as by curve-fitting the data for the individual.

Listed below are example formulas for global metrics for glucose, where A, B and C are weighting factors that can be either constants or derived functions. Other formulas for global metrics may be used, and one or more of these derived metrics may be shown on-screen in the software application used by the patient.

Global Metric 1=A*GV+B*GL+C*PPP

Global Metric 2=C*PPP/(A*GV+B*GL)

Global Metric 3=(A*GV)/(B*GL)+PPP

Global Metric 4=(A*GV+C*PPP)/B*GL

Global Metric 5=C*PPP/(A*GV+B*GL)

The derived functions for weighting factors A, B and C can be, for example, a polynomial function, exponential function, logarithmic function or power-law function. In some embodiments, a rate of change may be used as part of the functions, such as a rate of change of metabolic sensed values where rapid rise or rapid decrease of these values correspond to certain behaviors such as eating or exercise. For example, during rapid rates of change these weighting factors may increase how portions of the index (global metric) may be weighted, such as the post prandial peak value. The five examples of global metric calculations above use different additive and multiplicative combinations.

Each variable in the overall (global) metric is likely to be weighted differently for each individual. For example, GV is known to rise as a person goes from normal weight to overweight to obese, and higher values of GV are known to be correlated to those who are overweight. Thus, for higher weight individuals, the weighting factor “A” for GV in the global metric of the present embodiments may be higher than for people with lower or normal weight. In another example, the PPP is often more muted in the morbidly obese population than people in the overweight category, and thus the weighting factor “C” of PPP in the global metric of the present embodiments may be lower in value for morbidly obese patients compared to overweight individuals. In yet another example, the GL may be more correlative for weight gain or loss in the normal population rather than in the overweight or obese populations. Consequently, GL may have a higher weighting factor “B” for individuals in lower weight categories. Note that these examples describe general trends, which may not apply to every case since the actual weighting factors for each situation is highly individualized. Furthermore, although these examples show how a person's weight category can be used to affect the derived functions or rate of change for the weighting factors, other aspects may be used to tailor the weighting factors.

The rate of change can also differ widely in different cohorts. For example, for PPP a fast rate of change may potentially result in the PPP being more correlated to weight gain even though the PPP value is low. The rate of decline from a PPP may be especially relevant to long term weight gain with a slow decline more likely to be correlated to weight gain.

The present glucose variation monitoring and weight loss systems and methods integrate continuous glucose monitoring with, in some embodiments, image and auditory recognition software to provide information in a single displayed screen that predicts an individual's post-prandial glucose and guide food selection. The system receives meal inputs from the user to input what is being eaten. Then using analytics, which may be cloud-based, the system generates a series of parameters for the meal. Based on the meal parameters and the CGM measurements, the system calculates and displays actionable targets for the patient that are communicated back to the patient and displayed as behavior modification recommendations.

In some aspects of the present disclosure, measurements of metabolic indicators other than or in addition to glucose data are combined to provide further characterization of an individual's health characteristics. Each person's body behaves differently in response to eating certain foods and to physical activity, and systems and methods of the present disclosure can provide customized analyses and recommendations suited for each individual. During exercising, glucose levels can be an indicator of aerobic activity. Glucose levels typically increase initially and then decrease later after glucose is burned off. Ketone levels typically increase rapidly during exercise and then decrease (e.g., drop or decay), where the rate of decrease may be different from glucose. The level of ketones can be used to determine when the body is in a fat burning mode in which the body tries not to deplete short-term stores of muscle glycogen or blood glycogen. Lactate levels may rise during exercise, indicating muscle fatigue. Ketone is byproduct of fat metabolism, and lactate is the anaerobic energy pathway. Combined together, glucose, lactate and ketone describe the primary energy sources for the body.

Although continuous glucose monitors are common, conventional systems do not provide continuous monitoring of ketone or lactate. Systems and methods of the present disclosure advantageously consider at least two of glucose, ketone, and lactate or all of these quantities, beneficially providing more comprehensive and more accurate recommendations for people to address their weight loss, blood sugar, physical fitness, and other health monitoring needs. Embodiments utilize an insight recognized in the present disclosure that monitoring other metabolic indicators in addition to glucose can result in more complete, customizable metrics and recommendations for an individual and their specific needs and situations.

Glucose, lactate, and/or ketone responses to food and physical activity vary from person to person. For example, lactate may be a more important indicator of health parameters for one person while ketone may be more important for another person. Furthermore, the type of data such as a load or a variability, and/or certain relationships between the quantities (e.g., ratios or various combinations of the quantities) for characterizing a person's response will vary from person to person. In aspects of the present disclosure, systems and methods learn what relationships between the metabolic quantities best predict the person's response to food consumption and/or physical activity and provide recommendations suited for that person.

In some aspects, systems and methods calculate, using a processor, a global metric that involves different indicators of a metabolic substance or indicators from different types of metabolic substances. The global metric can be based on one or more of i) glucose data and ketone data, ii) the glucose data and lactate data, iii) the ketone data and the lactate data; or iv) the glucose data, the ketone data and the lactate data. Each of the data can include one or more type of measurements. For example, the glucose data may include a glucose variability (GV), a glucose load (GL), and/or a post-prandial peak (PPP). The ketone data may include a ketone load (KL) and/or a ketone variability (KV). The lactate data may include a lactate load (LL) and/or lactate variability (LV). The load is a cumulative amount, which may be determined by tracking the level of a substance over time and integrating an area under the curve of the tracked results. Some embodiments may include using the values of the levels (i.e., level of the substance at the current time). The systems and methods can determine what combination of these various quantities best represents the individual's response to certain food and/or physical activity.

In some cases, a ratio of the quantities (i.e., indicator ratio) may be used in calculating a global metric. An indicator ratio may include two of the following: glucose data, the ketone data, and the lactate data. For example, an indicator ratio may involve a ratio of glucose data and ketone data; i.e., a “GK” ratio. The GK ratio may be, for instance, one of: GK Ratio 1=GL/KL (ratio of loads), GK Ratio 2=GV/KV (ratio of variability), GK Mixed Ratio 1=GL/KV, or GK Mixed Ratio 2=GV/KL (ratio of variability of one substance to the load of another substance). An indicator ratio may involve a ratio of lactate data and glucose data; i.e., an “LG” ratio. The LG ratio may be, for instance, LG Ratio 1=GL/LL (ratio of loads), LG Ratio 2=GV/LV (ratio of variability), LG Mixed Ratio 1=GL/LV, or LG Mixed Ratio 2=GV/LL. An indicator ratio may involve a ratio of lactate data and ketone data; i.e., an “LK” ratio. The LK ratio may be, for instance, LK Ratio 1=LL/KL (ratio of loads), LK Ratio 2=LV/KV (ratio of variability), LK Mixed Ratio 1=LL/KV, or LK Mixed Ratio 2=LV/KL. The inverse of any of these ratios may also be utilized. For example, GK Ratio 1 may be the GL divided by the KL or the KL divided by the GL. As another example, LG Mixed Ratio 1 may be the GL divided by the LV or the LV divided by the GL.

In some cases, calculating correlations between the quantities may include a rate of change (ROC), such as a ketone rate of change (KROC), glucose rate of change (GROC) and/or a lactate rate of change (LROC). In one example, at least one of the KROC and the LROC is used in determining an individualized metric. The rate of change can provide additional insight into the patient's response behavior and can be used for improving the accuracy of tailoring a global metric for an individual.

In some cases, the global metric may involve various combinations of ketone data, where the combinations may use weighting factors designated as A and B below. Examples of ketone global metrics may include:

Global Metric K1=A*KV+B*KL;

Global Metric K2=(A*KV)/(B*KL);

Global Metric K3=(A*KL)/(B*KV);

Global Metric K4={(A*KL)/(KROC)}*Ketone Ratio, where the Ketone Ratio is a running ketone value (i.e., ketone level at a point in time) divided by an average daily ketone level.

In some cases, the global metric may involve various combinations of lactate data, where the combinations may use weighting factors designated as A and B below. Examples of lactate global metrics include:

Global Metric L1=A*LV+B*LL;

Global Metric L2=(A*LV)/(B*LL);

Global Metric L3=(A*LL)/(B*LV);

Global Metric L4={(A*LL)/(LROC)}*Lactate Ratio, where the Lactate Ratio is a running lactate value (i.e., lactate level at a point in time) divided by an average daily lactate level.

In some cases, the global metric may involve a mix of glucose data, ketone data and/or lactate data, where the combinations may use weighting factors designated as A, B, C and D below. Examples of global mixed metrics include:

Global Mixed Metric 1=A*GV+B*KV+C*GL+D*PPP

Global Mixed Metric 2=A*GV+B*KV+C*KL+D*PPP

Global Mixed Metric 3=A*GL/KL

Global Mixed Metric 4=A*GV/KV

Global Mixed Metric 5=A*GROC/KROC

Global Mixed Metric 6=A*GL/KL+B*GV/KV+C*GROC

Systems and methods may calculate some or all of the above global metrics (e.g., metrics for one substance, mixed metrics, and/or indicator ratios) and determine which one(s) most accurately predict the user's trends, such as their weight loss, sugar levels, or other health monitoring aspects. For example, in a learning phase all of most of the global metrics (e.g., the ones most suitable for the user's goals) may be calculated, and then as the system continues in a monitoring phase, the system may focus on certain metrics and/or may identify other metrics to try based on historical trends of that individual. The system then can apply the learning to suggest practices for the user to implement in real time and/or in the future. In some examples, the system may focus on a particular aspect such as weight loss or fitness benefits (e.g., based on instructions from the user on what their goal is, or based on orders from a physician), and the system can determine which global metric(s) are best suited for that goal. For instance, the system may use a ratio of glucose data and ketone data to target weight loss, where the system may provide recommendations on keeping the ratio below or above a certain value. In another case, a ratio involving lactate data and glucose data may be more suited for targeting health fitness goals.

In some examples, the weighting factors in the global metrics may be chosen based on the person's body type (e.g., normal, overweight, obese). For instance, a weighting factor may be chosen to emphasize a certain quantity more or less (e.g., ketone, lactate, glucose or a combination of any of these) depending on if the person is in a normal weight range, overweight or obese. In other examples, the weighting factors may be chosen based on other factors such as existing medical conditions, ethnicity, family history, or other aspects that relate to trends in how that individual might respond to food or exercise. The weighting factors A, B, C or D for each corresponding metabolic indicator may be adjusted by the system (e.g., which weighting factors to use and what value to assign to those weighting factors) according to the user's situation.

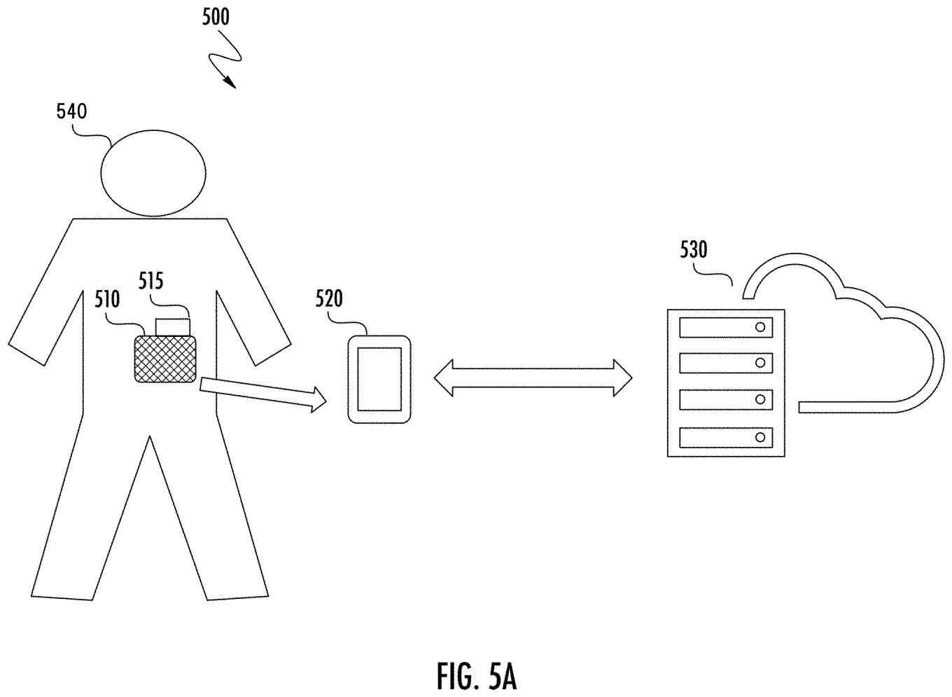

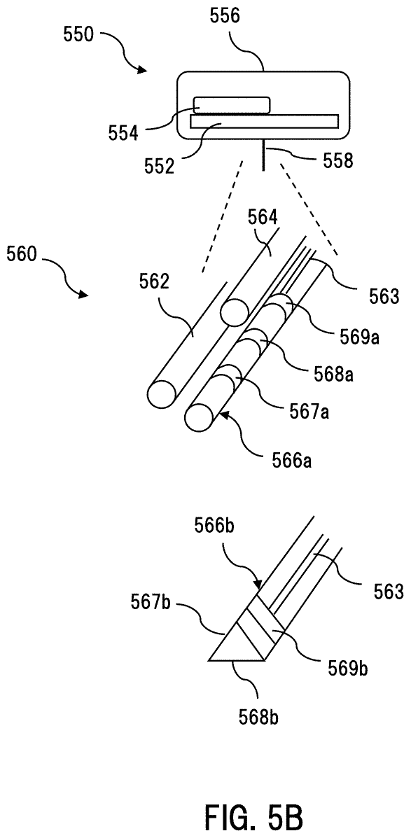

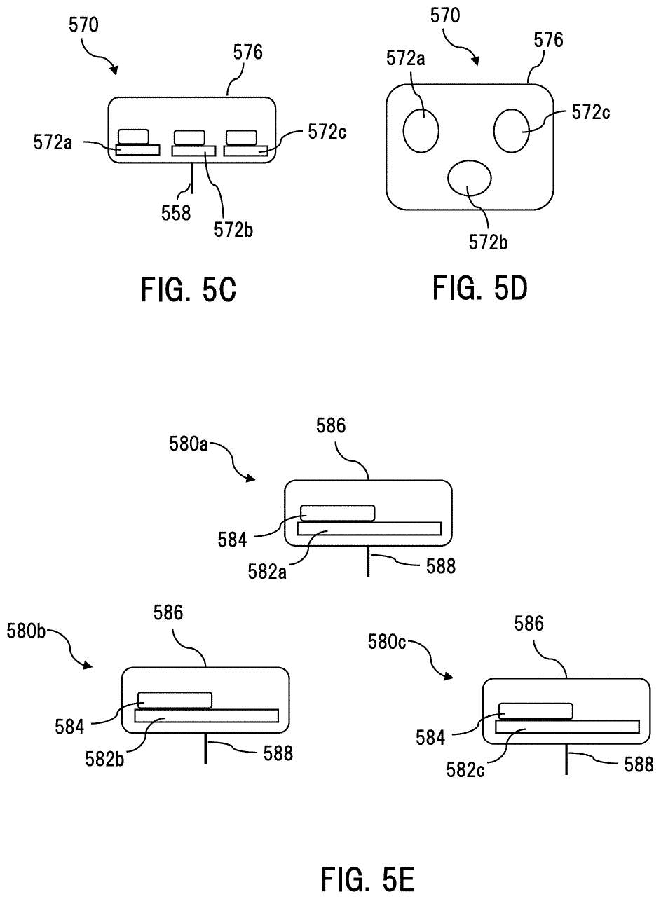

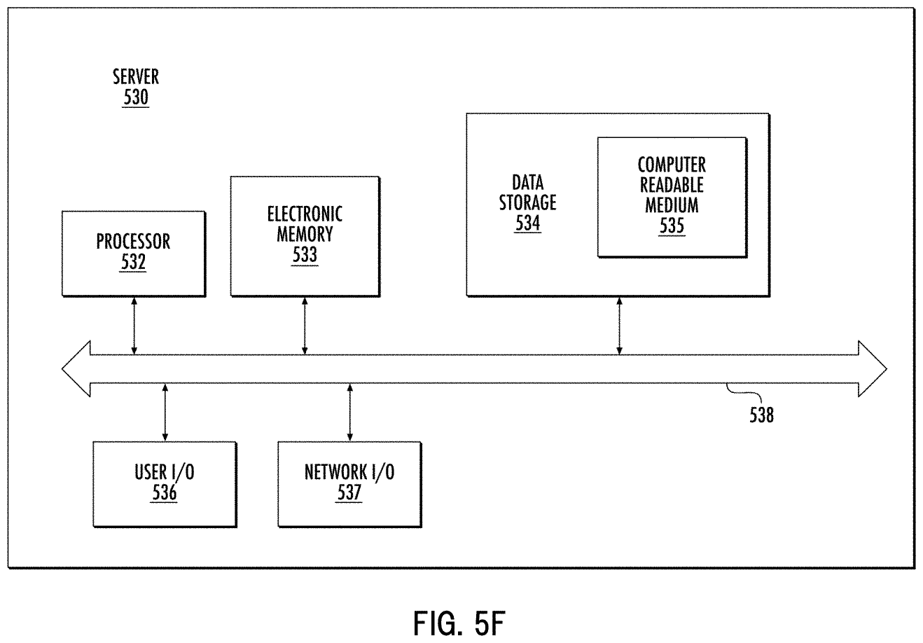

FIG. 5A is a schematic of a metabolic monitoring system 500 which includes a metabolic monitoring device 510, an electronic device 520 and a server 530, which is depicted as being cloud-based. The metabolic monitoring device 510 can be a CGM sensor for monitoring glucose, or can be a device that measures glucose as well as other metabolic characteristics such as ketones, lactates, and/or fatty acids. For example, the metabolic monitoring device 510 can represent the use of multiple types of sensors or can represent a single sensor that is configured to measure multiple types of substances. The metabolic monitoring device 510 and typically, a wearable patch, may be applied to the patient 540 by an applicator, where the metabolic monitoring device 510 takes glucose and/or other metabolic readings from under the surface of the patient's skin. The metabolic monitoring device 510 may be connected to an electronics unit 515 in the wearable patch, and the electronics unit 515 is configured to transmit metabolic data readings wirelessly to an electronic device 520. Electronic device 520 may be, for example, a mobile device such as a smartphone, a tablet, or smart watch, or a laptop computer. In some embodiments, the electronic device 520 is not mobile but may be, for example, a desktop computer or medical equipment configured to receive readings from the metabolic monitoring device 510 via the electronics unit 515.

The device 520 receives food intake information (e.g., food eaten during or between meals) from the patient 540, and the food information and glucose readings are transmitted to the server 530. The transmission may be accomplished through a variety of paths, communication access systems or networks. The networks may be the Internet, a variety of carriers for telephone services, third-party communication service systems, third-party application cloud systems, third-party customer cloud systems, cloud-based broker service systems (e.g., to facilitate integration of different communication services), on-premises enterprise systems, or other potential data communication systems. The server 530 can represent a cloud-based processing system. In other embodiments, the meal and glucose data can be stored and processed on the device 520 itself, such that the server 530 is not required.

FIGS. 5B-5D are schematics of metabolic monitoring devices that may be implemented as the metabolic monitoring device 510, in accordance with embodiments. In these examples, the metabolic monitoring devices are configured to take measurements of multiple metabolic indicators, such as glucose, ketone, and/or lactate.

FIG. 5B is a simplified schematic of a metabolic monitoring device 550 in which a sensor assembly 560 has a single component that takes measurements of more than one metabolic indicator, such as at least two of glucose, ketone and lactate. Metabolic monitoring device 550, shown in a side cross-sectional view, is a wearable device, such as by adhering it to the user's skin. An electronics board 552, connected to sensor assembly 560, is powered by a battery 554. Both the electronics board 552 and battery 554 are enclosed in a housing 556. In some cases, the electronics board 552 may in the form of a printed circuit board assembly onto which sensor wires, a transmitter, a microprocessor, and other associated components are mounted as disclosed in U.S. Pat. No. 11,478,174, entitled “Continuous Glucose Monitoring Device,” which is owned by the assignee of the present disclosure and is hereby incorporated by reference.

When the device is in use, a needle 558 is deployed from the surface of the housing 556 that is adjacent to (e.g., adhered to) the user's skin, penetrating the skin to sample the user's interstitial fluid and/or blood. The sensor assembly 560 is routed through the needle 558 and implanted underneath the skin of the user. In the example of FIG. 5B, sensor assembly 560 is a three-electrode design (i.e., 3-pole) that includes a reference electrode 562, a counter electrode 564, and a working electrode 566a (i.e., working wire). In other examples, the sensor assembly 560 may be a two-electrode design involving the reference electrode 562 and working electrode 566a but not a counter electrode 564.

Working electrode 566a has three active areas in this illustration, a glucose active area 567a, a ketone active area 568a and a lactate active area 569a. The active areas (i.e., active/sensing region or zone) configured for sensing a substance, such as having coatings or layers of electrochemical sensing materials suited for detecting the metabolic substance. In this manner, all three metabolic substances may be measured by the working electrode 566a. Although the glucose active area 567a is shown nearest the tip of the working electrode 566a, followed by the ketone active area 568a and the lactate active area 569a, the active areas may be arranged in other orders. Furthermore, in other examples only two of the active areas may be included (e.g., glucose and ketone, or glucose and lactate, or ketone and lactate). Materials for each of the active areas may be electrodeposited onto a core working wire which may be, for example, gold-, platinum- or carbon-based.

FIG. 5B also depicts a working electrode 566b which is an alternative configuration for the working electrode 566a. Working electrode 566b comprises a wire having a triangular cross-section rather than a round cross-section as in working electrode 566a. For the triangular working electrode 566b, each flat face is used for sensing a different substance. For example, active area 567b on one face of the triangular wire may be coated with a material for sensing glucose. Active area 568b may be configured to sense ketone, and active area 569b may be configured to sense lactate. In some examples, only two faces of the triangular wire may be utilized as active areas (e.g., for glucose and ketone, or glucose and lactate, or ketone and lactate). In operation, the working electrode 566b is utilized with reference electrode 562 and optionally counter electrode 564. Materials for each of the active areas may be electrodeposited onto a core working wire which may be, for example, gold-, platinum- or carbon-based.

In the device 550 which uses a working electrode having multiple active areas (sensing regions), electrical signals from each of the active areas may be sent to electronics board 552 via conductive traces along the working electrode. As an example, a conductive trace 563 may be a stripe, such as a carbon-based screen-printed stripe, along the length of the electrode wire. In FIG. 5B, conductive trace 563 is shown only for active areas 569a and 569b, for simplicity of illustration. However, each active area would have a corresponding conductive trace 563, which ensures that each active area remains electrically independent from each other and therefore can sense independently. In some embodiments, economies of scale in sensor production may be achieved by manufacturing a working electrode that is suitable for all product variations (e.g., having glucose, ketone, and lactate sensing areas). Then for specific products, electrical connections may be made to power and communicate only certain zones (e.g., conductive traces to one, two or three of the active areas).

In embodiments, the active areas may be fabricated using new coating technology similar to 3D printing. Embodiments may utilize aqueous-based enzyme formulations for the glucose, ketone and lactate. In one example, a high viscosity polyurethane or acrylic formulation may be used to deposit a small amount of the enzyme of choice to the active region or zone via 3D printing. The deposited material is then cured/dried to form the active area. In another example, an ultraviolet (UV) or light-based (e.g., laser) crosslinking system is utilized to crosslink a silicone-, polyurethane-, or acrylic-based formulation having low viscosity, where the UV/light source is used to build one or more layers on the active region, similar to a resin-based 3D printing methods. In yet another example, a dual nozzle 3D printer may be utilized with an ionic-based crosslinking system in which an enzyme solution is deposited with a first nozzle, then a crosslinking solution is deposited with second nozzle to crosslink the system.

In the configuration of FIG. 5B, the sensor assembly 560 is depicted as a multi-region, single working electrode wire where the sensing regions (active areas) have a shared reference electrode and electronics. In other embodiments, more than one reference electrode may be included, with one reference electrode corresponding to each active area 567a-b, 568a-b and 569a-b.

FIG. 5C shows a side view and FIG. 5D shows a bottom view of a metabolic monitoring device 570 that includes three separate sensors in housing 576, in accordance with embodiments. In FIG. 5D, the device may have a width and a length of 10 mm to 30 mm each, such as being a 20 mm by 20 mm square in one example. Metabolic monitoring device 570 includes a glucose sensor board 572a, a ketone sensor board 572b, and a lactate sensor board 572c, each with corresponding sensor wires (not shown). Description of the electronics board 552 in FIG. 5B applies to the sensor boards 572a-b-c. The sensors from all three sensor boards 572a-b-c may be routed through needle 558 such that a single needle insertion can achieve multiple types of metabolic readings. In some embodiments, only two types of sensors may be included in the metabolic monitoring device 570 (e.g., glucose and ketone, or glucose and lactate, or ketone and lactate).

FIG. 5E shows another embodiment in which separate sensor packages (devices) may be worn by a user. In such an implementation, the metabolic monitoring device collectively includes more than one sensor package, for instance two or three sensor packages where a different metabolic substance is monitored by each sensor package. In FIG. 5E, a continuous glucose monitoring device 580a (CGM), a continuous ketone monitoring device 580b, and a continuous lactate monitoring device 580c are shown. Each device is configured similar to the sensor assembly 560 of FIG. 5B, having an electronics board (glucose detection electronics board 582a, ketone detection electronics board 582b, or lactate detection electronics board 582c) and a battery 584 enclosed in a housing 586. Each electronics board 582a-b-c and sensors (not shown) are configured to sense its corresponding substance. A user may wear one or more device in a mix and match manner (e.g., one, two or all three devices 580a-b-c in any combination) depending on what metabolic indicator(s) they wish to monitor and depending on what their health goals are. In some examples, the devices 580a, 580b, and/or 580c may be in wireless communication with each other and with a central processor, such as to coordinate timing of taking measurements.

FIG. 5F is a simplified schematic diagram showing an embodiment of server 530 of FIG. 5A (representing any combination of one or more of the servers) for use in the system 500, in accordance with some embodiments. Other embodiments may use other components and combinations of components. For example, the server 530 may represent one or more physical computer devices or servers, such as web servers, rack-mounted computers, network storage devices, desktop computers, laptop/notebook computers, etc., depending on the complexity of the metabolic monitoring system 500. In some embodiments implemented at least partially in a cloud network potentially with data synchronized across multiple geolocations, the server 530 may be referred to as one or more cloud servers. In some embodiments, the functions of the server 530 are enabled in a single computer device. In more complex implementations, some of the functions of the computing system are distributed across multiple computer devices, whether within a single server farm facility or multiple physical locations. In some embodiments, the server 530 functions as a single virtual machine.

In the illustrated embodiment, the server 530 generally includes at least one processor 532, a main electronic memory 533, a data storage 534, a user input/output (I/O) 536, and a network I/O 537, among other components not shown for simplicity, connected or coupled together by a data communication subsystem 538. A non-transitory computer readable medium 535 includes instructions that, when executed by the processor 532, cause the processor 532 to perform operations including calculations of global metrics, determining of an individualized metric, and providing behavior modification recommendations as described herein.

In accordance with the description herein, the various components of the system or method generally represent appropriate hardware and software components for providing the described resources and performing the described functions. The hardware generally includes any appropriate number and combination of computing devices, network communication devices, and peripheral components connected together, including various processors, computer memory (including transitory and non-transitory media), input/output devices, user interface devices, communication adapters, communication channels, etc. The software generally includes any appropriate number and combination of conventional and specially-developed software with computer-readable instructions stored by the computer memory in non-transitory computer-readable or machine-readable media and executed by the various processors to perform the functions described herein.

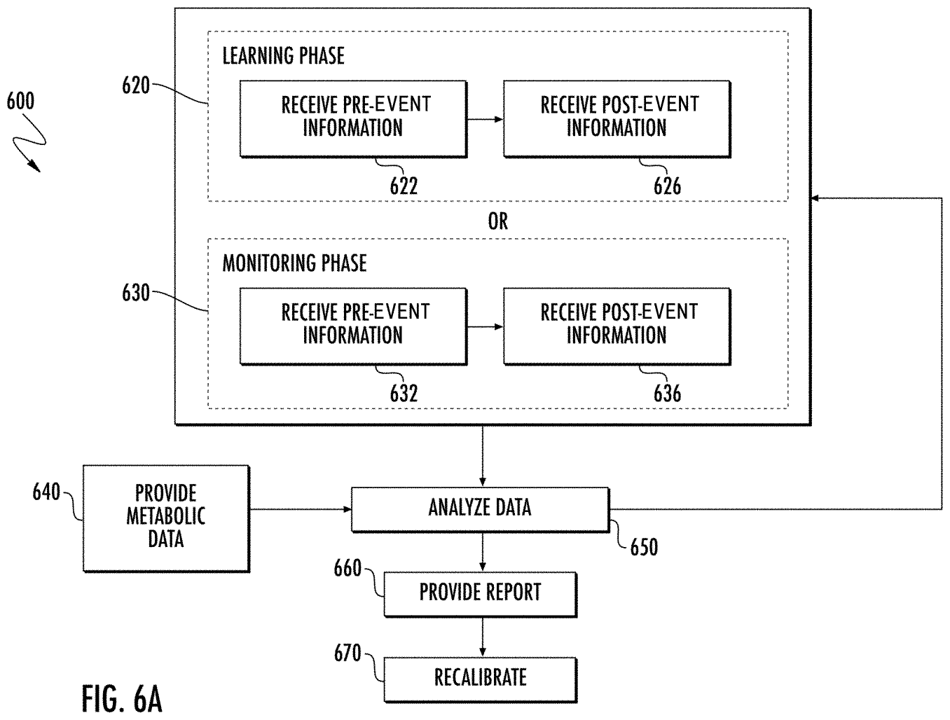

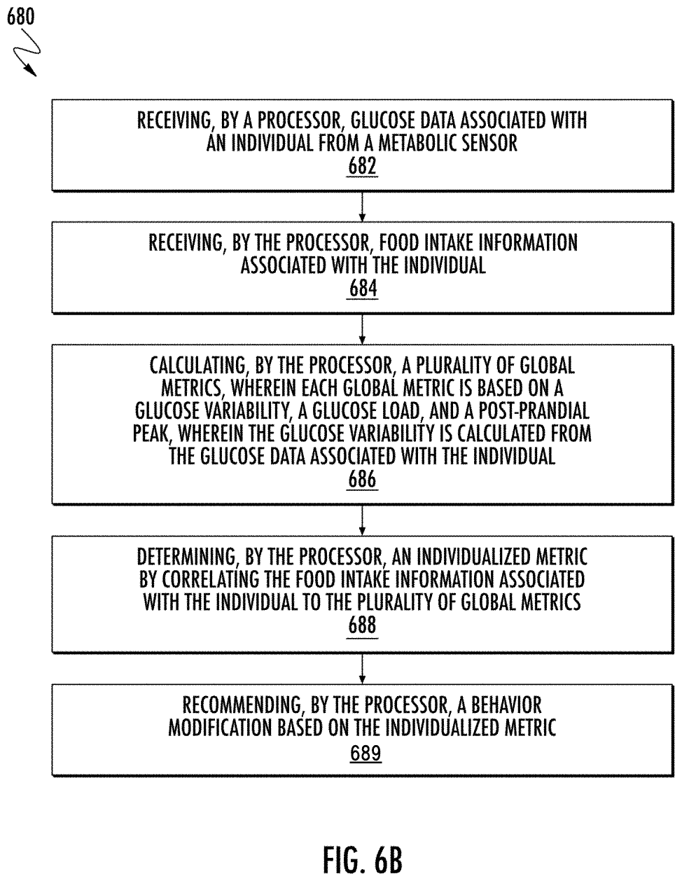

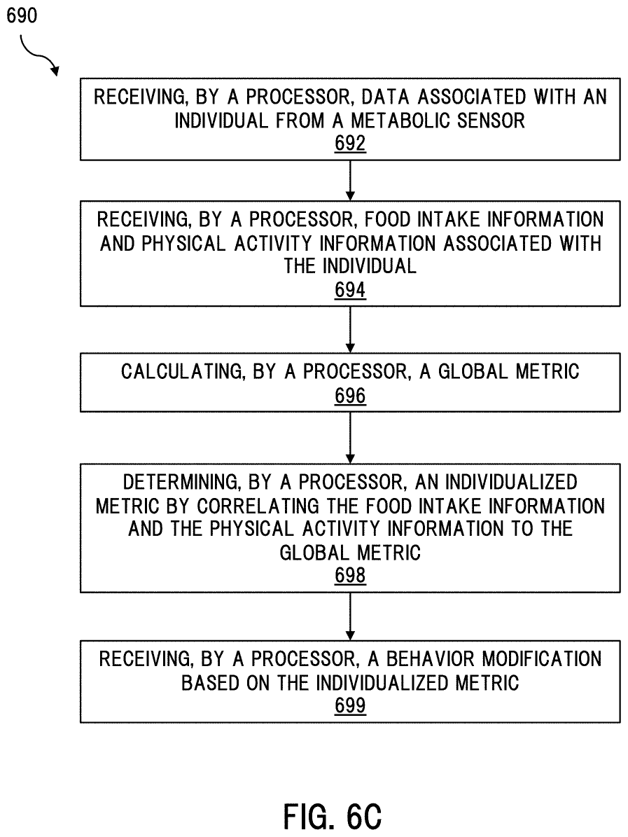

FIG. 6A is a flowchart showing a method 600 of monitoring metabolic activity, in accordance with some embodiments. The metabolic activity may include changes in level, load, peak and/or variability for one or more quantities (e.g., glucose, lactate, ketone and/or fatty acids) in response to an event or action such as consuming food and/or performing physical activity. The steps of the method 600 may be implemented on a non-transitory machine-readable medium, such as a software application on a computer processor. The method 600 begins with a learning phase 620 in which pre-event information (e.g., before eating and/or before physical activity such as exercising) is received by the system in block 622. After the system is trained to recognize typical inputs by the individual, the system performs the monitoring phase 630 which involves receiving pre-event information in block 632 and receiving post-event information in block 636.

In various embodiments, food intake information can be input in blocks 622, 626, 632 and/or 636 by one or more methods such as uploaded images or photographs, audio (e.g., voice) input, video recordings, or typed text on the device or other input system. In various embodiments, physical activity information can be input by a user (e.g., by typing text or orally dictating information), by fitness types of software applications and devices, or other input methods. Physical activity information can include, for example, heart rate, number of steps, type of activity, the patient's weight, and/or estimated calories used. In some embodiments, the inputs may be entered or supplied by a third-party such as a software application. As an example, the information may be open source shared via a health app on the user's mobile phone. In another example, the user may input the event information (e.g., food intake information, or type of exercise and duration) into the software application, and the data is uploaded to the system by the software application.

In some embodiments in which eating is the event, the system can use image recognition and/or voice recognition for identifying the food intake information that is received from the user, such as identifying a food item and an amount of the food item consumed. For example, before eating, blocks 622 and/or block 632 may involve uploading a picture of what is to be consumed as well as receiving a verbal entry about the food. The patient then consumes the food. After eating, the system receives food information in block 626 and/or block 636 which can include receiving another picture along with a verbal estimate of the percentage of the total food that was consumed. If there is insufficient information received, the system may prompt the user to enter the missing information. For instance, the system may determine from the post-meal photo that there has been a decline in food present and can request verbal entry of the amounts and/or types of food consumed.

In some embodiments in which physical activity is the event, the user may input one or both of the pre-event information (blocks 622 and 632) and the post-event information (blocks 626 and 636). For example, the individual may enter complete information on their workout regimen before or after performing the workout. Alternatively, the individual may input their starting information on the physical exercise in blocks 622 or 632 (e.g., start time of a run or swim) and then provide ending information (e.g., end time and/or distance of the run or swim) in blocks 626 or 636. In various embodiments, information on the physical activity may include the type of exercise, duration in time, amount (e.g., distances, weights, repetitions), level of rigor (e.g., high-intensity cardiovascular, low impact, heart rates), or any combination thereof. In some examples, the pre- and/or post event information on the physical activity can be uploaded by other sensors such as a smart device (e.g., smart watch, smart ring, mobile phone) or by a separate software application linked to the metabolic monitoring system, where these input sources can provide data on heart rate, speed, calories burned, and the like.

For either the learning phase 620 or monitoring phase 630, in block 640 the metabolic data including glucose, ketone, and/or lactate data is provided by the metabolic (e.g., CGM) sensing device. In block 650 the system, such as the server or the device, analyzes the data—that is, the food information, physical activity information, and the metabolic readings from the metabolic monitoring device. The food information can include the types of food, amounts, and sequence in which the food items were eaten. Food information can include any item consumed by the individual such as beverages, vitamins, and energy supplements. Physical activity information can include examples described throughout this disclosure such as amounts, durations, levels, and types of exercise. Additional data analyzed by the system may include the sequence and timing between food consumption, physical activity events, and the metabolic readings (e.g., how long after one event did a second event occur or was a metabolic measurement taken). As one example, if the system sees a delay time between when the glucose is low and the ketone is high (e.g., peaks), then the system might determine that individual has entered a fat burning stage.

Individual metrics may be generated and displayed for the patient and are based on the calculated global metrics described herein. In some embodiments, the global metrics are based on glucose variability using formulas that combine GV, GL and PPP using weighting factors depending on each individual. In some embodiments, global metrics use a combination of two or more of any of the following: GV, GL, PPP, KV, KL, LV, LL, GROC, KROC and LROC, where the combinations may use weighting factors. Displayed metrics may also include possible rates of metabolic change.

The system predicts responses by the individual, such as the patient's PPG and whether the meal will be in a high, borderline, or safe zone for the particular patient. These PPG zones may be indicated visually on the display of the device by, for example, red, yellow, and green colors, respectively. Determination of which global metric to use as the individualized metric to display for a patient can be based on factors such as their weight category, the presence of a diabetic condition, or their individual historical trends. For example, the determining of the individualized metric may include learning from the received historical food intake information associated with the patient and historical metabolic index calculations. A behavior modification recommendation is generated by analyzing correlations between the global metric and food intake, where the analysis may use artificial intelligence (AI) in some embodiments.

An example of a behavior modification recommendation based on the metrics is suggesting an order of eating foods in a meal, such as eating protein or fat first to produce lower GV and PPP for a particular person. In another example, an individual may have high glucose responses to certain foods (i.e., carbohydrates), and recommendations can be made by the system to substitute foods that result in a lower response. These substitutions could be alternative types of food items or could be another food within the same food type, based on the individual's own data on how they respond to each type of food eaten. Over time, the system's response database (e.g., data storage 534 of FIG. 5B) of the patient's glucose responses and food intake information grows and more correlations are gained, and better advice on food substitutions becomes available to the user. The individual response to food and food groups is not static and changes over time as the individual loses weight, so constant updating of the response database is performed by the system. In some embodiments, meal and exercise timing can also be correlated to help the individual produce lower metrics (GV and GL in particular) in order to produce weight loss and to sustain weight loss in the individual.

In the learning mode, the cycle of learning phase 620, block 640 and block 650 then repeats so that the system can learn the patient's typical metabolic responses. Once there is a reasonable match between the predicted and actual results the learning phase is complete. The learning phase can also be used to train the analysis system on voice recognition, such as of audio input of food intake information from the individual. In the monitoring mode, the cycle of monitoring phase 630, block 640 and block 650 repeats to provide monitoring of the individual and to provide recommendations such as for addressing whether the user is losing weight with their current behavior.

The patient continues to use the application in a monitoring phase 630. The system receives pre-event information (e.g., pre-eating or pre-physical activity information) in block 632 (e.g., by receiving an uploaded picture of what is eaten), and receives post-event information (e.g., post-eating information or post-physical activity information) in block 636 after the food is eaten or the activity is completed. As described in relation to learning phase 620, in some embodiments the system uses a mobile device input (e.g., by smart phone) of food intake or physical activity via photos and/or voice-driven inputs to obtain caloric estimates and other data. Receiving food and/or physical activity information from other devices is also possible, such as by a desktop computer that can then send the information to a mobile device or to a computer server that has the metabolic readings.

For calculations during the monitoring phase, in block 640 glucose data is again provided to the system by the metabolic (e.g., CGM) monitoring device 510 via the electronics unit 515. In block 650 the system analyzes the data (e.g., food information such as a meal, drink, or snack) and the metabolic readings (e.g., glucose readings from the CGM) to correlate event information to the global metrics. The system can then calculate a prediction of a metabolic response range (e.g., glucose level zone) the patient will be in. The system may be configured to recognize possible errors. For example, if there is a spike in glucose level without food data entry, the system requests entry of the information. The predictions can be performed in real-time, thus providing useful information for the user to monitor their metabolic and alter their behavior immediately as needed. Metabolic sensors may continuously measure and track the patient for a desired time period, such as several days (e.g., up to 14 days). The process of analyzing the data in block 650 continues during this time period, using the input information from the monitoring phase 630 and the metabolic data in block 640.

The analysis during the monitoring phase 630 may continue to use the individualized metric that was determined during the learning phase 620 or may change the individualized metric to adapt to changes in the user's response. Changing the individualized metric may involve adjusting the weighting factors and/or changing which global metric to use for the individualized metric. In some embodiments the behavior in the monitoring phase 630 may be different than in the learning phase 620 due to information regarding meals not being received. In such cases, the system sends reminders to the patient that data is not received and suggests repeat CGMs.

In step 660 a report is generated periodically (daily, for example), that provides information such as the mean glucose level, number of spikes, highest spike, foods that caused spikes, and the like. The report may also include similar data for ketone and lactate relative to physical activity and/or food intake. The displayed information may be generated as an aggregate value (day by day, weekly, etc.) or for each individual meal or activity. This information can be presented visually, such as percentages of meals in red, yellow, or green zones, where the zone categories are based on which global metric is used to serve as the individualized metric. The reports may include a daily predicted mean glucose and other metrics that a user may want to monitor. The reports may convey a behavior modification recommendation based on the individualized metric. Behavior modification recommendations can include at least one of a type of food to eat, a sequence in which to eat different food types, a timing of meals during a day, a timing of exercise in relation to a meal or exercise. In block 670, recalibration of the entire system can be suggested with repeat metabolic monitoring at regular intervals.

Systems and methods may determine recommendations based on various factors. For example, when a user is doing their daily routines, the systems and methods may suggest changes in: diet, timing of food intake during the day or relative to other activities, an order of food intake in meals (e.g., protein, fat, and carbohydrates), and/or an exercise parameter (e.g., exercise type, level, timing, duration). Since conventionally there are no existing continuous data streams for lactate and ketone, embodiments of the present disclosure provide new insight into the body's metabolism. In an example experiment performed in relation to this disclosure, by wearing a continuous lactate sensor and adjusting intensity in an experimental subject's workouts to see rises above basal lactate levels, the subject was able to both lose weight and improve cardiovascular fitness, to achieve optimal performance.

The present methods and systems may use machine learning algorithms such as supervised and/or unsupervised learning. Supervised learning may involve providing known inputs (e.g., recognition of food input data from audio input or photo images) or known responses to exercise or food consumption based on other users in a database. In unsupervised learning, the system may learn how to classify and predict inputs and metabolic responses of the individual without previously known data.

Methods and systems of the present disclosure uniquely combine metabolic indicators of different types to provide more complete information on a patient's behavior than can be assessed conventionally with just glucose monitoring or with one-time measurements of ketone or lactate. Some embodiments utilize metabolic data such as ketone and lactate to make recommendations to a user to adjust their eating and/or physical activity behavior. Using multiple metabolic indicators, particularly by continuously monitoring the indicators (e.g., ketone and/or lactate), is beyond what conventional systems can provide since typically only glucose is monitored on a continuous basis. Lactate meters exist (e.g., for athletes or to diagnose sepsis) but only take measurements at a certain point in time. Similarly, ketone meters exist (e.g., for helping in weight loss management) but only take discrete measurements. The use of a continuous metabolic monitoring device that can provide measurements of multiple metabolic indicators offers more comprehensive insight and analysis on behavior modifications than can be provided by existing systems.

As described herein, other quantities can be measured in addition to glucose. For example, sensors for lactate, ketone, etc., can be utilized. These additional sensors can be separate sensors from the glucose sensor or can be combined into a single device with the glucose sensor (e.g., as described in FIGS. 5B-5D) to provide the metabolic data in block 640 to be used in the analyses. The additional sensors can help indicate further aspects of a person's metabolic response, such as during exercise. For example, higher ketone levels indicate more fat burning, and lactate levels indicate a shift between aerobic and anaerobic activity. Accordingly, additional metrics calculated and displayed to the patient may also include direct ratios of multiple metabolites such as glucose to ketone/lactate/free fatty acid or calculated metabolic indices such as glucose indices to ketone/lactate/free fatty acid indices. Correlations can be created between meal input ratios and these indices to generate individualized expert advice. In some embodiments, tracking of these additional aspects may be useful for athletes in determining a training program.

The meal parameters used in the analyses can include ratios of estimated carbohydrates, proteins, and fat content, as well as approximate caloric portion size. The system may request several mixed meals like a protein bar to sample a broad array and to provide better machine learning. These meals are then indexed along with metabolic sensor metrics and tracked (e.g., in a cloud-based infrastructure) for the individual patient.

Metabolic sensor data in block 640 may be augmented by additional sensor data such as heart rate, blood pressure, steps, weight, and/or accelerometer activity and sleep monitors, all of which may be transmitted to the mobile device, such as wirelessly. These measurements can be used to create correlations to the overall metrics as well. Aggregate data from the metabolic sensor (e.g., glucose, ketone, free fatty acid, etc.) and response to all activities (e.g., meals, sleep, exercise, general levels of activity) can have additional cross-correlations with heart rate, blood pressure, activity and other physical sensors included in the system and recorded in the databases.

FIG. 6B is a flowchart showing a method 680 of monitoring metabolic activity using glucose data, in accordance with some embodiments. At block 682, a processor receives glucose data associated with an individual from a metabolic sensor. At block 684, the processor receives food intake information associated with the individual. At block 686, the processor calculates a plurality of global metrics. Each global metric is based on a glucose variability, a glucose load, and a post-prandial peak. The glucose variability is calculated from the glucose data associated with the individual. At block 688, the processor determines an individualized metric by correlating the food intake information associated with the individual to the plurality of global metrics. At block 689, the processor recommends a behavior modification based on the individualized metric. The behavior modification may include recommendations on a food parameter, such as types of food to eat or not eat, an order of eating certain foods (e.g., proteins, carbohydrates, fats), amounts of food, and timing of when to eat during the day or before/after other activities.

FIG. 6C is a flowchart of a method 690 of monitoring metabolic activity, in accordance with some embodiments. At block 692, a processor receives data associated with an individual from a metabolic sensor, the data comprising at least two of glucose data, ketone data and lactate data. At block 694, the processor receives food intake information and physical activity information associated with the individual. At step 696, the processor calculates a global metric. In some examples, the global metric is based on a global metric that is based on i) the glucose data and the ketone data, ii) the glucose data and the lactate data, iii) the ketone data and the lactate data; or iv) the glucose data, the ketone data and the lactate data. In some examples, the global metric is an indicator ratio comprising at least two of the glucose data, the ketone data, and the lactate data. In some examples, the processor calculates a plurality of global metrics and chooses a global metric that best matches or predicts the individual's responses. At block 698, the processor determines an individualized metric by correlating the food intake information and the physical activity information to the global metric. Block 699 involves recommending, by the processor, a behavior modification based on the individualized metric. The behavior modification may include recommendations on a food parameter, such as types of food to eat or not eat, an order of eating certain foods (e.g., proteins, carbohydrates, fats), amounts of food, and timing of when to eat during the day or before/after other activities.

In embodiments of FIG. 6C, the glucose data comprises a glucose variability (GV), a glucose load (GL), and a post-prandial peak (PPP); the ketone data comprises a ketone load (KL) and a ketone variability (KV); and the lactate data comprises a lactate load (LL) and lactate variability (LV). The global metric may comprise any of the various ratios described herein, such as a ratio chosen from a GK Ratio of the glucose data and the ketone data, an LG Ratio of the lactate data and the glucose data, or an LK Ratio of the lactate data and the ketone data. The global metric may be chosen from any of the global metrics described herein, such as a ketone global metric, a lactate global metric, or a global mixed metric. The calculating may comprise calculating a plurality of global metrics, and determining may comprise which global metric of the plurality of global metrics to correlate the food intake information and the physical activity information to. The data is metabolic data is provided by a continuous metabolic monitoring device. The behavior modification may comprise a food parameter or an exercise parameter.

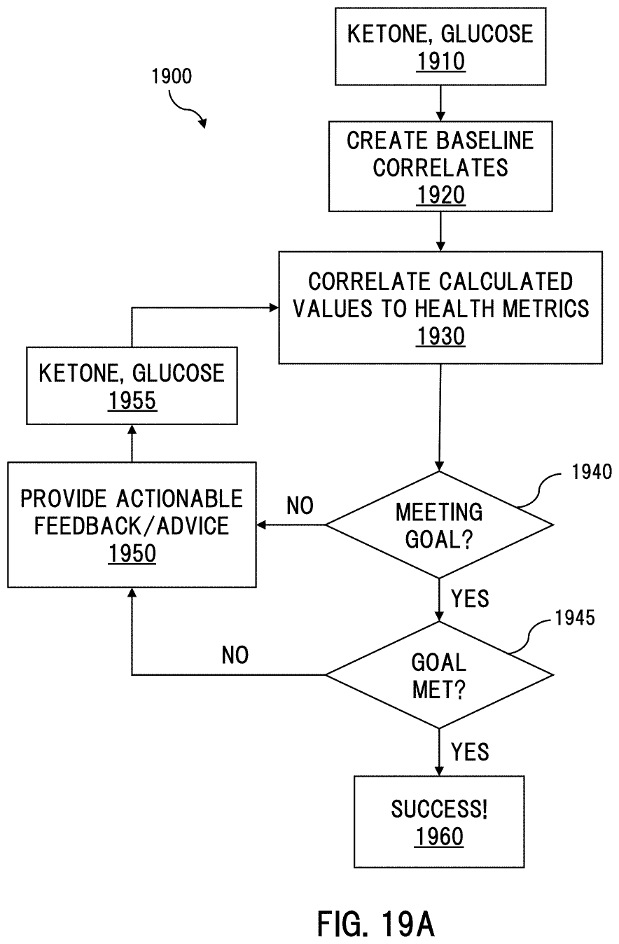

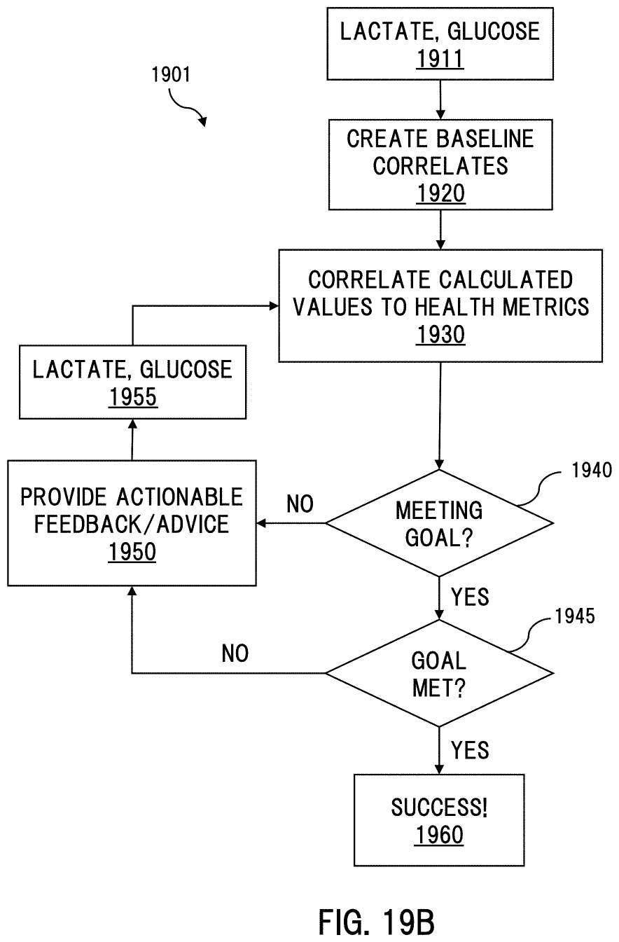

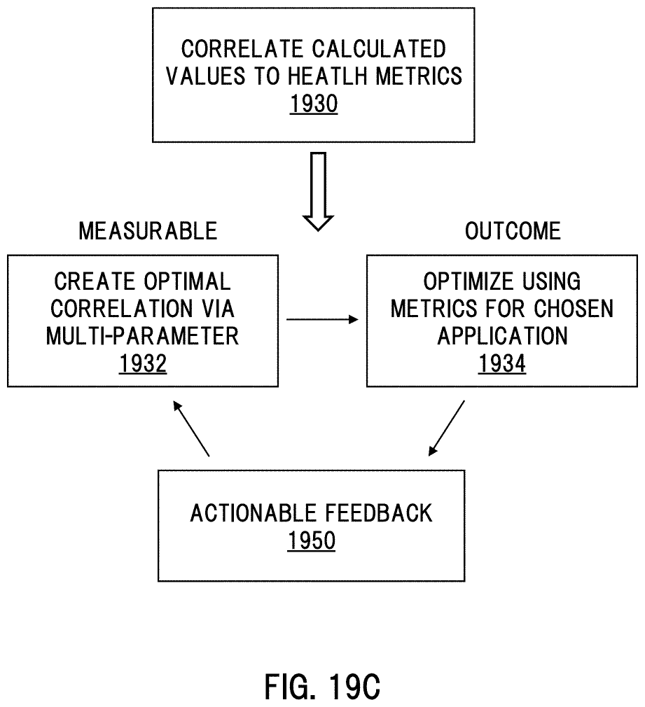

FIGS. 19A and 19B are flowcharts of methods for deciding what metrics to use for the individual and how to determine an individualized metric. First, the individual is queried on what overall goal they want such as weight loss, weight gain (e.g., increased muscle mass), improved fitness, or other goal. The methods then seed the analysis to find the best correlative measure for the individual for their chosen use. For example, the seeding may utilize as a starting point certain global metrics that are more suited for the particular goal, and weighting factors that are appropriate for characteristics of the individual (e.g., weight category). The methods then can refine what global metrics to use as an individualized metric for that person. FIG. 19A represents example methods for achieving weight loss, while FIG. 19B represents example methods for achieving improved fitness.

Method 1900 in FIG. 19A for achieving weight loss begins with block 1910 in which ketone and glucose measurements are taken by a continuous monitoring device (e.g., device(s) from FIGS. 5B-5D). In block 1920, baseline correlations are created, such as seeding the calculations with a GK ratio (e.g., GK Ratio 1 or 2, or GK Mixed Ratio 1 or 2). Block 1930 involves correlating calculated values to health metrics, such as open-source health metrics provided by the user's device (e.g., mobile phone, smart scale). At decision point 1940, the method 1900 determines whether the user's goals are on track; i.e., in this example, whether progress in weight loss is being made toward the user's end goal (e.g., achieving a target weight, or losing a target amount of pounds in a certain amount of time). If not, the method 1900 provides actionable feedback or advice in block 1950 (i.e., behavior modification recommendation as in block 689 and block 699 of FIGS. 6B and 6C, respectively) for improvement based on the metrics. The behavior modification may include a food parameter or an exercise parameter, for example, one or more of changing the types of foods to eat, changing the order of eating certain foods (protein, fat, carbs) to improve metrics, adjusting a type of exercise (and/or duration, intensity), or altering or other behaviors to increase ketone levels (fat burning). If progress is being made toward the user's goal at decision point 1940, the method determines at decision point 1945 whether the end goal has been met. If the end goal has been successfully achieved, the method is complete at block 1960. If not, actionable feedback or advice is again provided at block 1950. If goals are not met in either decision point 1940 or 1945, further ketone and glucose measurements are taken in block 1955, and the cycle of block 1930, decision points 1940 and 1945, and blocks 1950 and 1955 is repeated until success of meeting the overall goal is completed in block 1960.

Method 1901 in FIG. 19B for achieving fitness goals begins with block 1911 in which lactate and glucose measurements are taken by a continuous monitoring device (e.g., device(s) from FIGS. 5B-5D). In block 1920, baseline correlations are created, such as an LL/GL ratio. Block 1930 involves correlating calculated values to health metrics, such as open source health metrics by a user's device (e.g., smart watch, fitness monitor). At decision point 1940, the method 1900 determines whether the user's goals are on track; i.e., whether progress in fitness loss is being made toward the user's end goal (e.g., improving a resting heart rate, being able to travel a certain distance during exercising). If not, the method 1900 provides actionable feedback or advice in block 1950 (i.e., behavior modification recommendation as in block 689 and block 699 of FIGS. 6B and 6C, respectively) for improvement based on metrics. The behavior modification may include a food parameter or an exercise parameter, for example, for example, adjusting a type of exercise (and/or duration, intensity), changing when to exercise in relation to eating or other activities, or altering or other behaviors to adjust aerobic or anaerobic response to exercise. If progress is being made toward the user's goal at decision point 1940, the method determines at decision point 1945 whether the end goal has been met. If the end goal has been successfully achieved, the method is complete at block 1960. If not, actionable feedback or advice is again provided at block 1950. If goals are not met in either decision point 1940 or 1945, further ketone and glucose measurements are taken in block 1955, and the cycle of block 1930, decision points 1940 and 1945, and blocks 1950 and 1955 is repeated until success of meeting the overall goal is complete in block 1960.

FIG. 19C provides example details of the loop in FIGS. 19A and 19B that involve block 1930 for correlating calculated values to health metrics, such as open source health metrics. The correlating utilizes measurable quantities in block 1932 (e.g., glucose, ketone, lactate, fatty acids), in which the systems and methods create an optimal correlation via multi-parameter analysis. As an example, a three-parameter optimization may include maximizing ketone load, minimizing glucose variation, and achieving certain metabolic indicator ratios. In block 1934, outcomes are analyzed using the correlation from measurable quantities in block 1932. The outcomes in block 1934 may be metrics for the chosen goal/application (e.g., weight loss, fitness, or both) to optimize against. Example outcomes may include the person's weight, or a number of calories burned. Actionable feedback in block 1950 is then provided based on the status of the metrics in comparison to the desired outcome(s). Actionable feedback can include one or more of an order of food within a meal, timing of meals during the day or relative to other activities, timing of exercise during the day or relative to other activities, and an intensity of exercise. As measurements continue to be taken, the loop of blocks 1932, 1934 and 1950 (reflected by the loop of blocks 1930, decision points 1940 and 1945, and blocks 1950 and 1955 in FIGS. 19A-19B) is continued until the outcome goal is met.

FIG. 7 is a tree diagram that depicts many of the inputs that could be used by artificial intelligence to perform the correlation and trend analysis to create tangible advice for the individual. For example, some embodiments involve using AI heuristics for creating success profiles for population cohorts. The success profiles may include success behaviors, food intake, exercise, and other factors in a proven weight loss cohort. Some embodiments may include using AI data summaries/correlates to be sent to an insurer, clinician, dietician, etc., for review, help with compliance and expert advice. AI may also be used to send user prompts or advice based on a success cohort, such as suggesting certain behavior based on an individual's index value.

Displays of meaningful indices/correlates, such as those in the reports of step 660, may be displayed in a simple, graphical format. As described earlier, each individual has different metrics and correlates based on analysis of their data. Weekly or monthly data can be aggregated and trended along with expert advice or reports that can be given via a patient caregiver or consultation. Displaying data or the behavior modification recommendation in a simple, graphical format keeps the user up-to-date in real-time so an immediate action can be performed based on the individualized metrics.

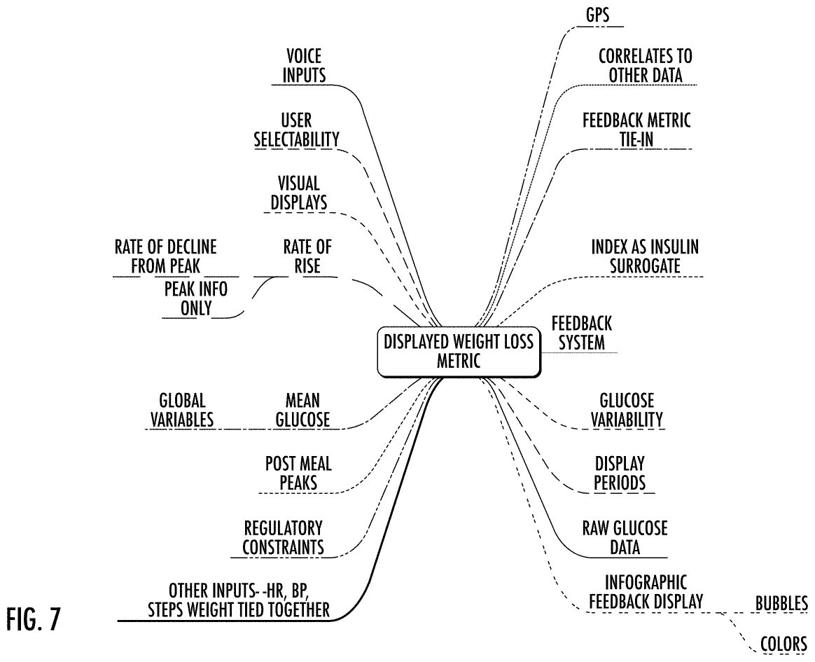

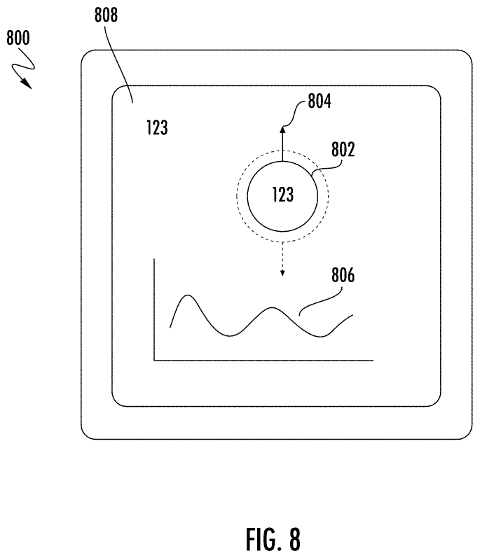

FIGS. 8-18 show examples of user interfaces that may be used to display metabolic values, global metrics, and recommendations for the user. Descriptions for glucose results in these figures may also be applied to ketone results, lactate results, or combinations thereof. Furthermore, descriptions of food intake recommendations in these figures may also apply to recommendations for exercise or other physical activity.

FIG. 8 shows an embodiment of a user interface 800 for the display of data. In this display screen user interface 800, a bubble style graphic 802 is utilized (showing an example value of “123”) that increases in size and changes color with the increase of the metabolic index (i.e., global metric). Additionally, this embodiment shows the use of an up or down arrow 804 to indicate changes from prior states. The display screen could be an initial display of this bubble style graphic 802 to provide a quick view of the global metric. Also shown in FIG. 8 is a graph 806 of a moving index to indicate GV values over time, and another index 808 (showing an example value of “123”) in the upper left corner which could be used, to show, for example, the number of calories. Other embodiments may include more in-depth analysis within layers of pull-down menus, and also feedback portions of the software application to provide directed advice. For example, the software application could include links to professionals such as a physician, dietician, or physical trainer. In various embodiments, the graphic 802, graph 806 and/or index 808 may be utilized to display any of the metabolic indices described herein such as glucose load, glucose variability, post-prandial peak, glucose rate of change, ketone load, ketone variability, ketone rate of change, lactate load, lactate variability, or lactate rate of change, or to display global metrics involving any of these quantities.

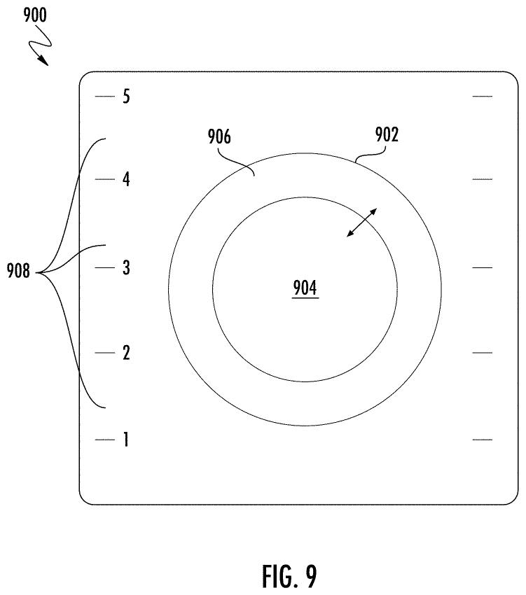

FIG. 9 is another embodiment of a user interface 900 for the display of data. This embodiment has a bubble 902 showing the current metabolic value 904 (e.g., glucose, ketone, or lactate), with the previous value indicated by bubble 906 concentrically displayed in a contrasting format, such as a ghosted format. The bubble 902 for the current value 904 and previous value in bubble 906 are sized to reflect their numerical values. The user interface 900 also displays a rate of change 908, embodied in FIG. 9 as a scale that may show a current value using, for example, highlighted numbers or a bar graph. In some embodiments, the user interface 900 may include more than one set of concentric bubbles and/or scales to display results for more than one metabolic value (e.g., glucose, ketone, or lactate) at the same time.

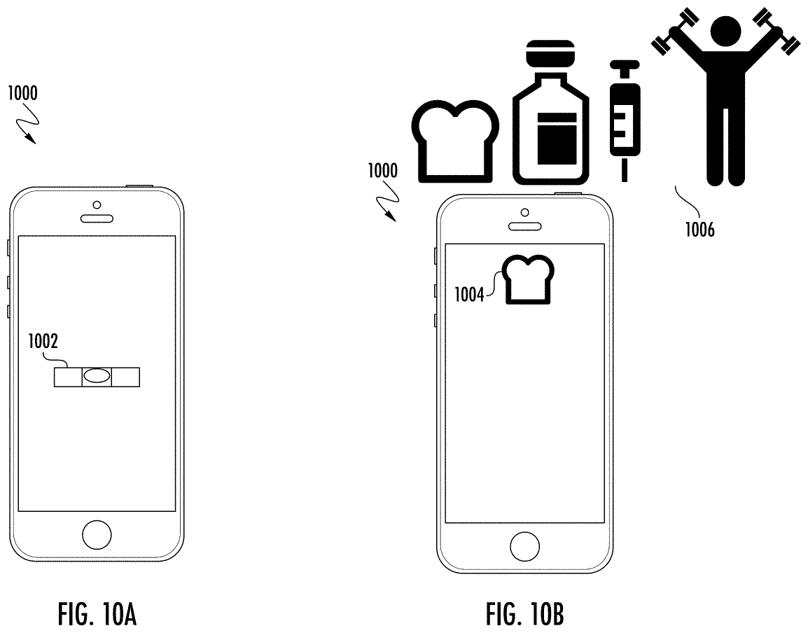

In some embodiments, the display of data may be a simple, visual cue for the user of the individualized metric such as the glucose level of the individual (or ketone level or lactate level). FIGS. 10A and 10B are embodiments of a user interface 1000 for the display of data, in accordance with some embodiments. In FIG. 10A, a level 1002—a bubble in a liquid that shows adjustment to a horizontal by movement of the bubble relative to a central zone—is shown which indicates in the same manner as a typical handyman tool. Data from the metabolic monitoring device 510 is transmitted to the device, and the user interface 1000 of the device visually indicates, for example, if the glucose value is high, low or within target by the level 1002. In this scenario as shown in FIG. 10A, the glucose value is level as indicated by the bubble between two vertical lines. However, if the glucose value is low, then the level 1002 would be displayed at an angle with the bubble to the left of the lower vertical line. In addition to the bubble in the level 1002 changing positions according to the glucose value, the level 1002 may change color such as green indicating an acceptable value, red indicating low and blue indicating high. In further embodiments, selecting the level 1002 when the glucose value is out of range, may launch a secondary display screen with recommendations of how to bring the glucose value back to within range (disclosed hereafter).

In FIG. 10B, the user interface 1000 depicts an icon 1004. The icon 1004 indicates an action to be taken. This may be helpful for the user not interested in numbers such as a child, or for international use, or any user who prefers a visual cue as a simple action to take when the glucose value is not within the acceptable range. For example, a slice of bread is shown which may be shown in different sizes to indicate “eat a snack” and how much to eat. A small icon 1004 of the slice of bread may be associated with a small snack. The icon 1004 may be chosen from a group of icons 1006 indicating to eat a snack, drink juice, take insulin or exercise. In other embodiments, more than one icon 1004 may be displayed at the same time depending on the data and what behavior modification is recommended.

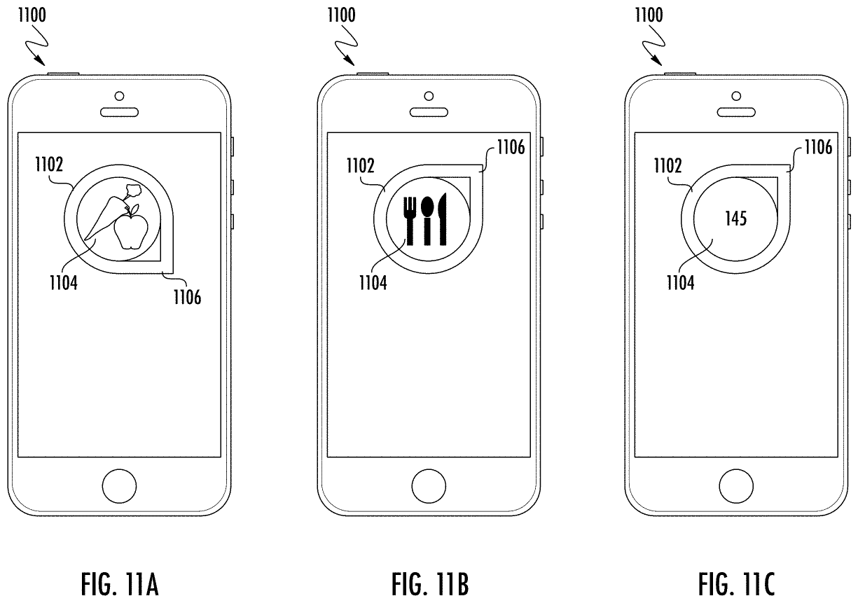

FIGS. 11A-11C illustrate user interfaces 1100 for the display of data, in accordance with some embodiments. The display shows a dial 1102 with an arrow 1106. With regard to the metabolic monitoring device 510 data and the global metrics, the dial 1102 may display a picture 1104 of the recommendation while the arrow 1106 may indicate the metabolic indicator value (e.g., blood glucose level) as low, thus the arrow 1106 pointing downward (FIG. 11A) or as high, thus the arrow 1106 pointing upward (FIG. 11B). By clicking on or selecting the dial 1102, more data may be displayed such as the actual glucose value as shown in FIG. 11C, or another metric such as the amount of carbohydrates that corresponds to the recommendation such as an apple picture 1104 in the dial 1102.

There are benefits to the user by displaying metrics on the user interface in a simple format in real-time. This reduces the burden of use on the user because the user can quickly understand if an action needs to be performed to modify their level of a metabolic substance. In some embodiments, the processor is part of a device having a display screen with a lock screen, home screen or wallpaper feature. The lock screen, home screen or wallpaper may be modified based on the glucose data associated with an individual from a metabolic sensor. In other words, the lock screen, home screen and/or wallpaper of the device may be changed based on the metabolic data from the sensor. For example, when a high glucose level (high load or high variation metric) is detected, the screen may change to a yellow screen. When a normal glucose level (normal load and variation metric) is detected, the screen may change to a green screen. When a low glucose level (low load and high variation metric) is detected, the screen may change to a red screen. A metric such as the glucose level and/or the behavior modification recommendation such as an action may also be communicated.

The described embodiment is a fast, discreet, convenient method for the user to understand the metric by merely glancing at the device (e.g., mobile phone, smart watch) without opening a software application. Moreover, this is different than receiving a notification on the home screen of the mobile phone because the present embodiments work with the operating system of the mobile phone and change the image of the lock screen, home screen and/or wallpaper based on the sensor data monitoring the user without user input. This occurs automatically and in real-time. For example, the user may opt-in to this feature in the software application. The software application may trigger a “software flag” based upon the user's data via the sensor. The software flag is transmitted to interact with the operating system or home screen setting of the operating system, and the lock screen, home screen and/or wallpaper then changes color and/or displays an image based on the user settings. This may be similar to settings or software applications that change the lock screen, home screen and/or wallpaper based on time.

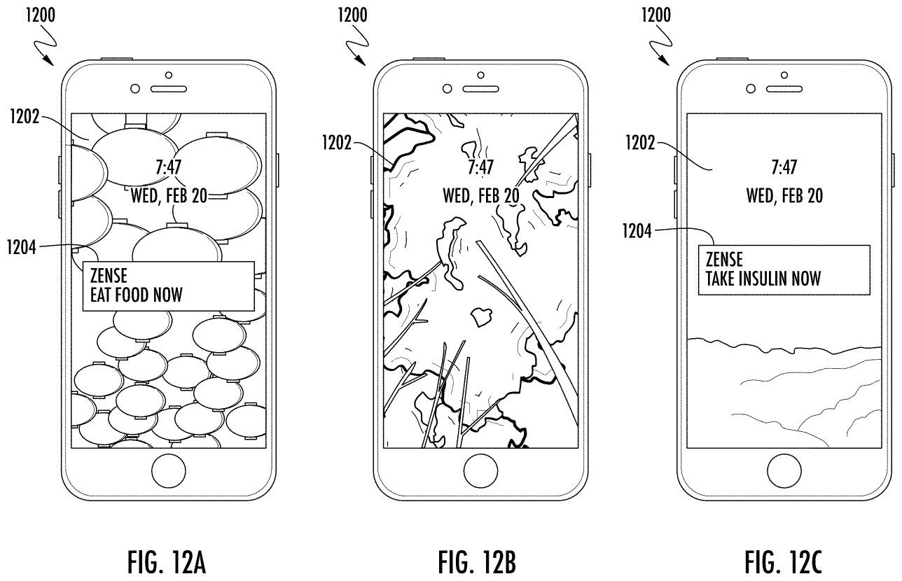

FIGS. 12A-12C depict embodiments of a user interface 1200 to communicate data, in accordance with some embodiments. The lock screen, home screen and/or wallpaper of the device, referred to as display screen 1202, may change color and/or display an image to indicate the level of a metabolic substance. In some embodiments, a user may choose a primary metabolic substance that they wish to focus on (e.g., glucose instead of lactate or ketone) to trigger changes in the home screen. FIG. 12A illustrates the display screen 1202 with an image of red flowers or lanterns which may indicate low blood glucose levels, FIG. 12B illustrates the display screen 1202 as green leaves on trees which may indicate an in-range blood glucose level, and FIG. 12C illustrates the display screen 1202 as a yellow sunset which may indicate a high blood glucose level. The colors and/or images of the display screen may indicate other data such as another metric or an action. This is a discreet way to communicate the status of the user's health without other people in the vicinity knowing what the colors or images represent.

The behavior modification recommendation may be displayed in a banner 1204 on the display screen 1202 that is the lock screen, home screen and/or wallpaper of the device. For example, based on the individual data of the user, FIG. 12A illustrates the display screen 1202 with a red image indicating a low blood glucose levels and the banner 1204 with the action of “eat food now,” and FIG. 12C shows the display screen 1202 with a yellow image indicating a high blood glucose and the banner 1204 with the action as “take insulin now.”

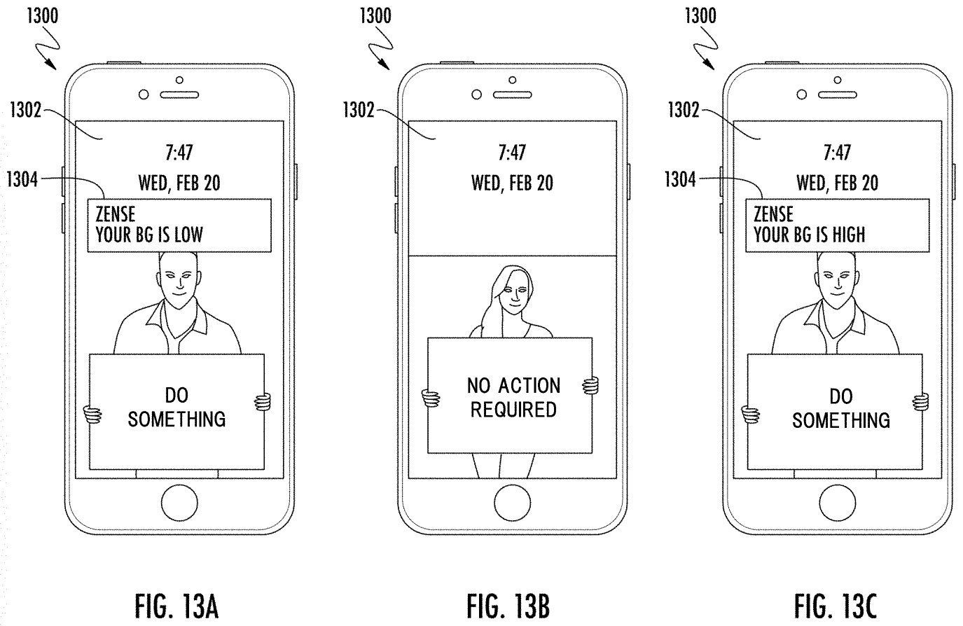

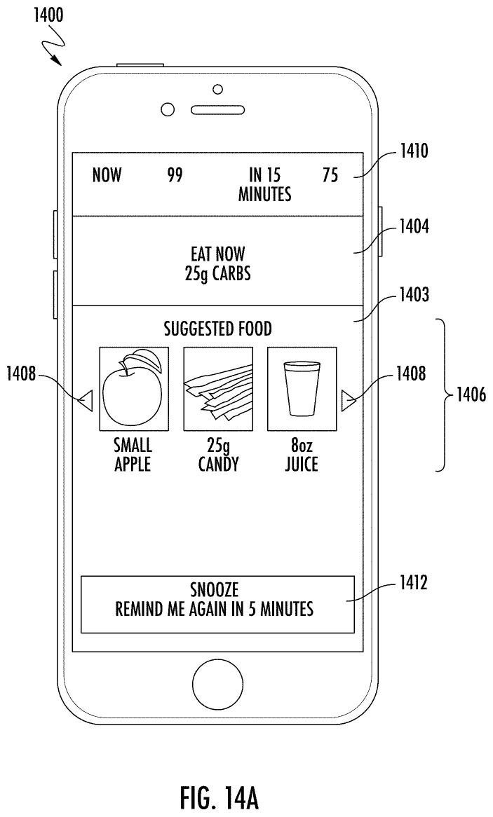

FIGS. 13A-13C depict other embodiments of a user interface 1300 to communicate data, in accordance with some embodiments. Similar to FIGS. 12A-12C, the display screen 1302 which is the lock screen, home screen and/or wallpaper of the device may change color and/or display an image to indicate the level of a metabolic substance. In this scenario, only two different colors may be used such as blue to indicate do something as in FIGS. 13A and 13C, and green in FIG. 13B to indicate the glucose level is in-range and no action is required. The banner 1304 on the display screen 1302 may indicate information about the metric such as the blood glucose is high (as shown in FIG. 13C) or the behavior modification recommendation such as to take medication (e.g., insulin) now.

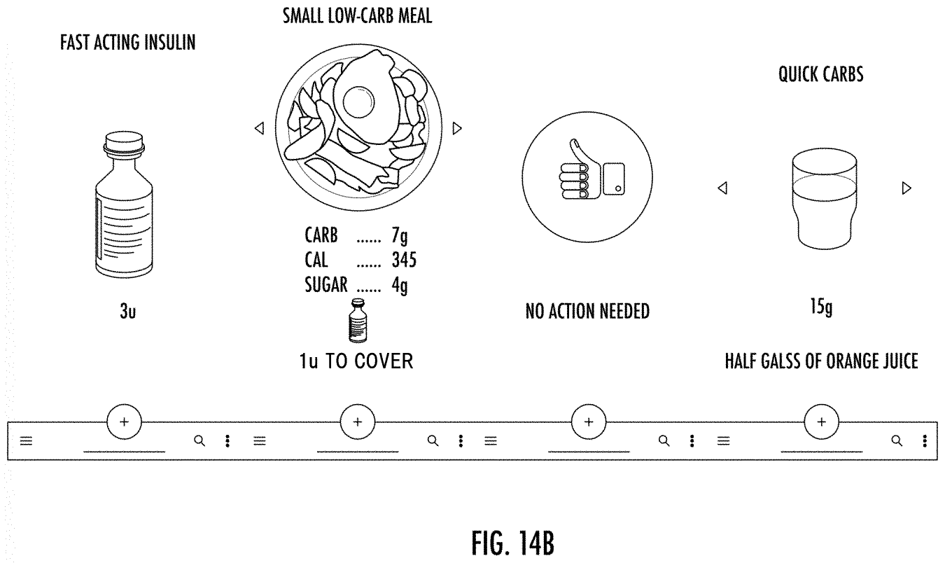

In some embodiments, more information or a deeper level of data may be needed to help guide the user. Referring to FIGS. 12A-12C, the user can click on the banner 1204 and a secondary user interface display screen opens. This may be an overlay on the lock screen, home screen and/or wallpaper, or a separate software application may open and the secondary screen is part of the software application. FIG. 14A shows an embodiment 1400 of a secondary user interface display screen 1403 to display data, in accordance with some embodiments. The banner 1404 repeats the action listed on banner 1204 and additionally, may recommend a specific amount or duration, e.g., in this case, grams of carbohydrates. This amount is what is needed in order to bring the user's blood glucose level back to into range. In section 1406, suggested foods are listed to achieve the recommended amount of carbohydrates. The suggested foods in section 1406 are not generic but specific candidates based on learning from historical food intake information received and historical metabolic index calculations. In some embodiments, the user can scroll through the suggested foods in section 1406 by clicking on arrows 1408. FIG. 14B shows examples of simple visuals of suggested foods, drinks or medications, in accordance with some embodiments, that may be shown in section 1406. Simple visuals with the number of units of carbohydrates listed may aid and train the user to understand what a specific amount of carbohydrates looks like.

A dashboard 1410 in FIG. 14A may list other metrics such as blood glucose stats for now and an estimate for 15 minutes later based on performing the recommendation such as eating a small apple. The estimate is also gleaned from historical food intake information received and historical metabolic index calculations since each individual has a different response to the same foods as demonstrated in FIG. 1. A footer 1412 may include a snooze function that can be selected by the user so that the user is reminded in a future amount of time such as 5 minutes. After the future amount of time, a visual or audio alert may be seen or heard.

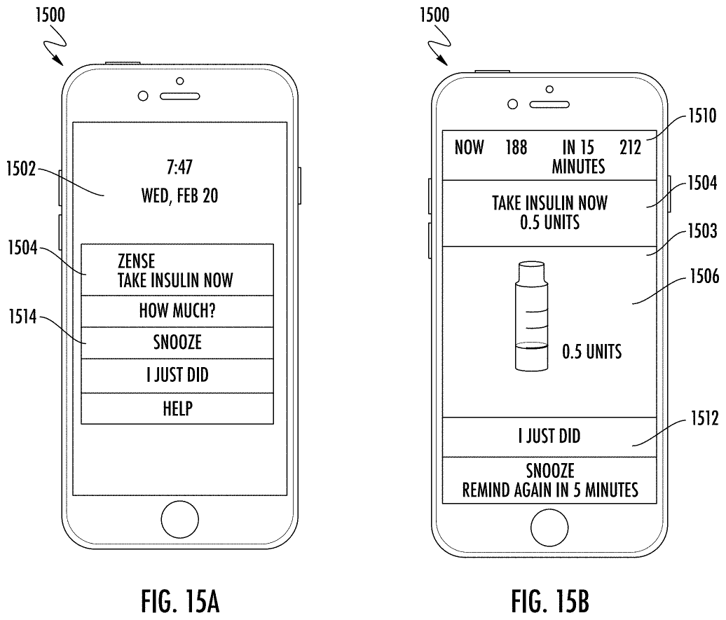

In another embodiment, FIG. 15A shows a user interface 1500 with a dropdown menu 1514 to further communicate data, in accordance with some embodiments. For example, if the user selects the banner 1504, the dropdown menu 1514 may appear as demonstrated in FIG. 15A. This may include another level of data such as a snooze feature on the display screen 1502 (as shown) or an amount or duration of the action displayed in the banner 1504. When the banner 1504 is selected again, the secondary user interface display screen 1503 opens. Screen 1503 is similar to the description of FIG. 14A. FIG. 15B is a secondary user interface display screen 1503 to display data, in accordance with some embodiments.

The banner 1504 repeats the action from the banner on the lock screen, home screen and/or wallpaper and additionally, recommends an amount of insulin to the consumer. This amount is what is needed in order to bring the user's blood glucose level back to in-range. In section 1506, a visual of the medication is shown. A dashboard 1510 may list other metrics such as blood glucose statistics for the present time and an estimate for in 15 minutes based on performing the recommendation such as consuming the insulin. A footer 1512 may include a snooze function that can be selected by the user so that the user is reminded in a future amount of time such as 5 minutes, or an option to confirm the action such as “I just did”. After the snooze amount of time, a visual or audio alert may be seen or heard.

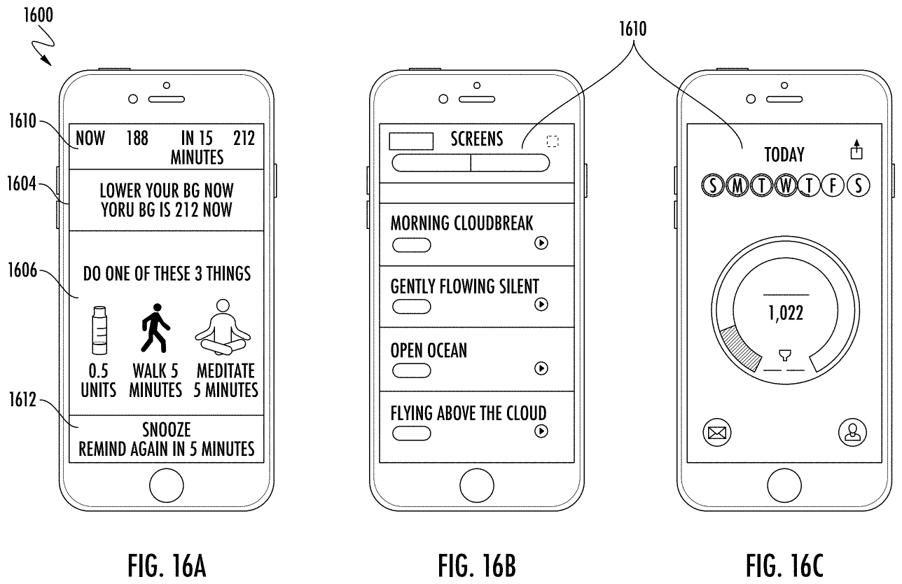

FIG. 16A is a user interface 1600 for the display of data, in accordance with some embodiments. The banner 1604, dashboard 1610 and footer 1612 are similar to the previous descriptions herein. In section 1606, suggested medication, activities or foods may be listed to achieve a blood glucose level within range. These are based on learning from historical food intake information received and historical metabolic index calculations for the specific individual. In some embodiments, by clicking on the visual in section 1606, a software application associated with the visual opens. In this way, when in the software application of the system, an unrelated-to-the-system second software application can be opened and accessed from the software application of the system. For example, by clicking on the meditation icon or the walk visual, an appropriate software application may open to track the data. These may be third-party software applications for exercise such as apps that track the daily number of steps taken per day. This data may be communicated to the system as feedback then used by the system for calculating and correlating metrics. FIGS. 16B and 16C depict example software applications in communication with the system, in accordance with some embodiments.

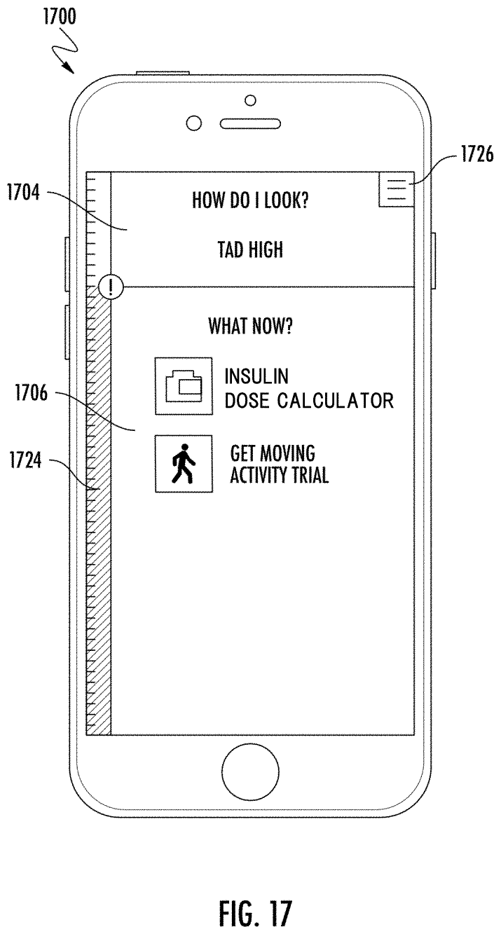

FIG. 17 is a user interface 1700 for the display of data, in accordance with some embodiments. The user interface 1700 may be on the lock screen, home screen and/or wallpaper, an overlay on the lock screen, home screen and/or wallpaper, or a separate software application. The banner 1704 communicates the status of a metric such as blood glucose. The section 1706 recommends the behavior modification such as medication, activities or foods to achieve a desired level of a metabolic substance (e.g., blood glucose level within a target range, ketone level or lactate level above or below a certain threshold). These are based on learning from historical food intake information received, historical responses to physical activities, and historical metabolic index calculations for the specific individual. A vertical bar indicator 1724 provides another visual cue of the metric. An options menu 1726 may include more in-depth analysis within layers of pull-down menus.

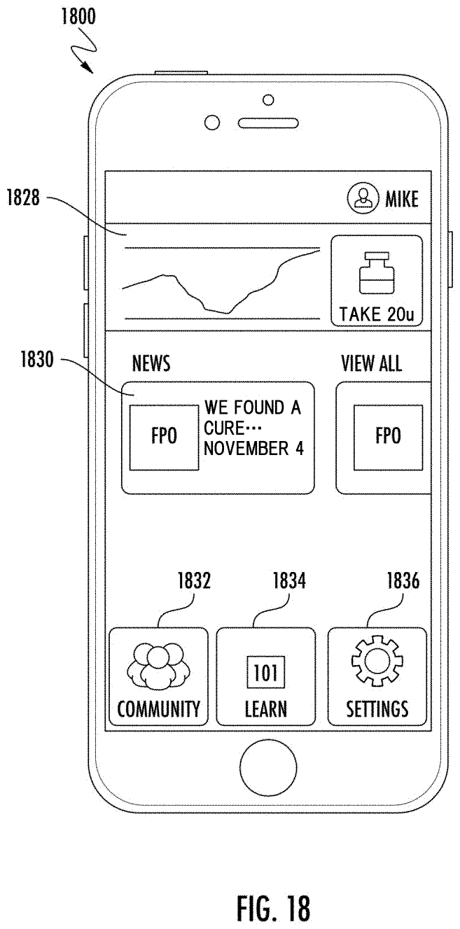

FIG. 18 is a user interface 1800 for the display of data, in accordance with some embodiments. As described with the various other example interfaces, the user interface 1800 may be on the lock screen, home screen and/or wallpaper, an overlay on the lock screen, home screen and/or wallpaper, or a separate software application. A status window 1828 communicates the status of a metric such as blood glucose as a graph over time without number labels. The graph terminates into a visual which is the behavior modification recommendation such as a medication, activities or foods to achieve a blood glucose level within range. The user interface 1800 may be more integrated with interests of the user by providing a media section with highlights and links to news articles 1830, community support 1832, training 1834 and settings 1836. By selecting the links of the media section, an unrelated-to-the-system second software application may open for the use of the user.

Some embodiments involve a system where understanding the individual's metabolic response to certain foods is used to guide the person to a metabolic data-driven weight loss program. For example, in some embodiments glucose (variation, total load and post-meal peaks) can be used for guiding weight loss. Further embodiments may use lactate sensing coincidently to distinguish exercise or other rises from food-related changes. Some embodiments may use a rate of decline in lactate levels from post-meal peaks to indicate fat storing.

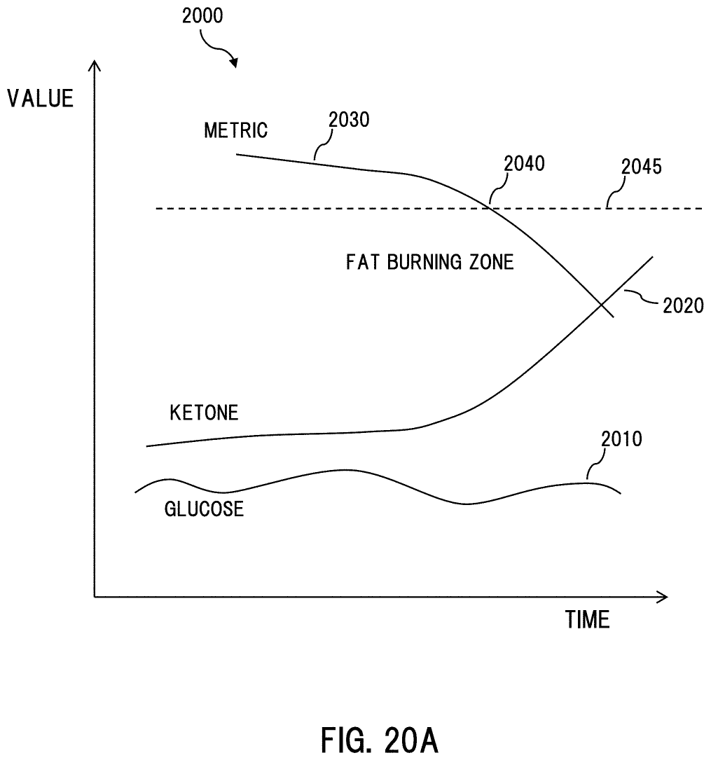

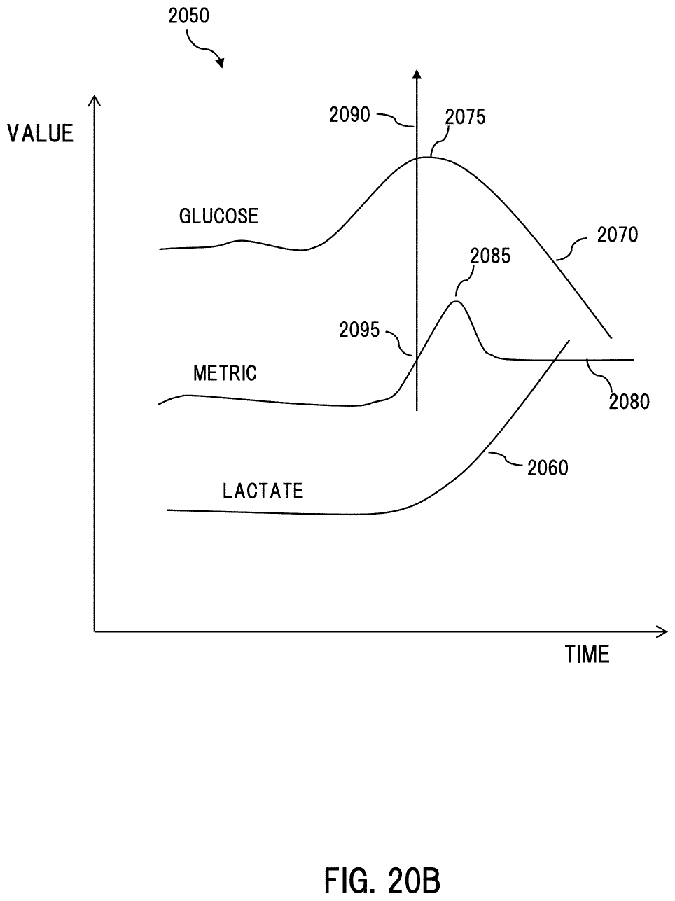

FIG. 20A is an example graph 2000 of utilizing an individualized metric for a person whose goal is weight loss, in accordance with embodiments. The graph 2000 represents values of a quantity on the Y-axis, as a function of time on the X-axis. During a period of stable, low glucose as seen in glucose curve 2010, the user starts to see a rapid rise in ketone levels as shown in ketone curve 2020. The metric utilized for the individual is a global metric chosen from the metrics disclosed herein and has values shown by metric curve 2030. In this example, the individualized metric utilizes glucose and ketone data that are continuously measured during the monitoring. The metric may involve, for example, dividing calculated quantities by the ketone level such that as the ketone levels rise, the calculated metric decreases. The system determines an inflection point 2040 where the metric curve 2030 crosses a fat burning threshold 2045 and then informs the user that they are in a fat burning zone. The longer the fat burning regime is maintained, the greater the potential weight loss is. The system can prompt the user when to create certain behaviors to initiate or extend the fat burning mode. The system can also suggest behaviors to avoid fat storing behaviors (where fat storing would have curves with opposite trends than what is seen in graph 2000) to create a net positive daily response (weight loss).

FIG. 20B is an example graph 2050 of utilizing an individualized metric for a person whose goal is fitness, in accordance with embodiments. The graph 2050 represents values of a quantity on the Y-axis, as a function of time on the X-axis. Lactate curve 2060, glucose curve 2070, and metric curve 2080 are shown, where the individualized metric is based on lactate and glucose values in this example. During a period of exercise, the glucose will initially rise as seen in glucose curve 2070, then reach an inflection point 2075 and fall, leading to a performance “bonk.” Lactate levels shown by lactate curve 2060 will concomitantly rise while the glucose levels are falling, where the rate of change of the lactate will be driven by the intensity of exercising. The system tracks the metric curve 2080, signaling the user at an actionable point 2090 which is prior to reaching a point 2085 where anaerobic metabolism takes over completely. Anaerobic metabolism can lead to performance suffering (e.g., activity deteriorating or person collapsing) and could also extend recovery from that point. The actionable point 2090 may be based on a trigger point 2095 which may be, for example, a threshold value for the rate of change in the metric. Based on the metric, the system will advise the user when to change their behavior (e.g., adjust intensity of physical activity, consume sugar/electrolytes/food) to avoid this anaerobic condition, and the system can provide recommendations to help optimize athletic performance.

Some embodiments may use global positioning system (GPS) data to provide supplemental information to the user. For example, based on an individual's GPS location, the system may provide locations of suggested restaurants and food recommendations served that the suggested restaurants. The system may also use location to encourage behavior modification, such as sending proactive texts or messages to not eat certain foods when the individual is identified as being in a location where that food is offered. GPS location data may also be used to map prior behaviors (e.g., food, others), or for “nagging” to prevent night binging. Another example of using GPS information in the present glucose monitoring and weight loss system includes using glucose peaks for retrospective understanding and behavioral monitoring.

Although embodiments have been described in relation to enhancing weight loss, the present glucose monitoring systems and methods can also be used to treat other diseases. For example, the present glucose monitoring systems and methods can be used for patients with cancer, from early stage to late stage, where the global metrics can be used to monitor food intake to reduce glucose variability. Glucose variability can uniquely serve as a proxy for insulin production and the presence of insulin-binding globulins and other potential growth-inducing factors that encourage cancer proliferation and increase the potential for recurrence or further metastases. In another example, the glucose monitoring can be used to address polycystic ovary syndrome, where modification of food intake can reduce glucose variability and insulin resistance, consequently improving fertility by increasing the chance of ovulation. Another example is the treatment of non-alcoholic fatty liver disease where glucose monitoring can prevent the elevation of glucose, which increases the deposition of fat in the liver that can then lead to, for instance, cirrhosis and liver failure.

Reference has been made in detail to embodiments of the disclosed invention, one or more examples of which have been illustrated in the accompanying figures. Each example has been provided by way of explanation of the present technology, not as a limitation of the present technology. In fact, while the specification has been described in detail with respect to specific embodiments of the invention, it will be appreciated that those skilled in the art, upon attaining an understanding of the foregoing, may readily conceive of alterations to, variations of, and equivalents to these embodiments. For instance, features illustrated or described as part of one embodiment may be used with another embodiment to yield a still further embodiment. Thus, it is intended that the present subject matter covers all such modifications and variations within the scope of the appended claims and their equivalents. These and other modifications and variations to the present invention may be practiced by those of ordinary skill in the art, without departing from the scope of the present invention, which is more particularly set forth in the appended claims. Furthermore, those of ordinary skill in the art will appreciate that the foregoing description is by way of example only, and is not intended to limit the invention.

What is claimed is:

1. A method comprising:
receiving, by a processor, data associated with an individual from a metabolic sensor, the data comprising at least two of glucose data, ketone data and lactate data;
receiving, by the processor, food intake information and physical activity information associated with the individual;
calculating, by the processor, a global metric that is based on i) the glucose data and the ketone data, ii) the glucose data and the lactate data, iii) the ketone data and the lactate data; or iv) the glucose data, the ketone data and the lactate data;
determining, by the processor, an individualized metric by correlating the food intake information and the physical activity information to the global metric; and
recommending, by the processor, a behavior modification based on the individualized metric.

2. The method of claim 1, wherein:
the glucose data comprises a glucose variability (GV), a glucose load (GL), and a post-prandial peak (PPP);
the ketone data comprises a ketone load (KL) and a ketone variability (KV); and
the lactate data comprises a lactate load (LL) and lactate variability (LV).

3. The method of claim 2, wherein the global metric comprises a GK Ratio of the glucose data and the ketone data, the GK Ratio being chosen from:
GK Ratio 1=GL/KL,
GK Ratio 2=GV/KV,
GK Mixed Ratio 1=GL/KV, and
GK Mixed Ratio 2=GV/KL.

4. The method of claim 2 wherein the global metric comprises an LG Ratio of the lactate data and the glucose data, the LG Ratio being chosen from:
LG Ratio 1=GL/LL,
LG Ratio 2=GV/LV,
LG Mixed Ratio 1=GL/LV, and
LG Mixed Ratio 2=GV/LL.

5. The method of claim 2, wherein the global metric comprises an LK Ratio of the lactate data and the ketone data, the LK Ratio being chosen from:
LK Ratio 1=LL/KL,
LK Ratio 2=LV/KV,
LK Mixed Ratio 1=LL/KV, and
LK Mixed Ratio 2=LV/KL.

6. The method of claim 2, wherein the global metric comprises a ketone global metric chosen from:
Global Metric K1=A*KV+B*KL,
Global Metric K2=(A*KV)/(B*KL),
Global Metric K3=(A*KL)/(B*KV), and
Global Metric K4={(A*KL)/(KROC)}*Ketone Ratio;
wherein the KROC is a ketone rate of change, the Ketone Ratio is a running ketone value divided by an average daily ketone level, and A and B are weighting factors.

7. The method of claim 2, wherein the global metric comprises a lactate global metric chosen from:
Global Metric L1=A*LV+B*LL,
Global Metric L2=(A*LV)/(B*LL),
Global Metric L3=(A*LL)/(B*LV), and
Global Metric L4={(A*LL)/(LROC)}*Lactate Ratio
wherein the LROC is a lactate rate of change, the Lactate Ratio is a running lactate value divided by an average daily lactate level, and A and B are weighting factors.

8. The method of claim 2, wherein the global metric further comprises a ketone rate of change (ROC) or a lactate ROC, wherein the ketone ROC or the lactate ROC is used in the determining of the individualized metric.

9. The method of claim 8, wherein the global metric comprises a global mixed metric chosen from:
Global Mixed Metric 1=A*GV+B*KV+C*GL+D*PPP,
Global Mixed Metric 2=A*GV+B*KV+C*KL+D*PPP,
Global Mixed Metric 3=A*GL/KL,
Global Mixed Metric 4=A*GV/KV,
Global Mixed Metric 5=A*GROC/KROC, and
Global Mixed Metric 6=A*GL/KL+B*GV/KV+C*GROC;
wherein A, B and C are weighting factors.

10. The method of claim 1, wherein:
the calculating comprises calculating a plurality of global metrics; and
the determining comprises which global metric of the plurality of global metrics to correlate the food intake information and the physical activity information to.

11. The method of claim 1, wherein the data is metabolic data is provided by a continuous metabolic monitoring device.

12. The method of claim 1, wherein the behavior modification comprises a food parameter or an exercise parameter.

13. A method comprising:
receiving, by a processor, data associated with an individual from a metabolic sensor, the data comprising at least two of glucose data, ketone data, and lactate data;
receiving, by the processor, food intake information and physical activity information associated with the individual;
calculating, by the processor, a global metric, the global metric being an indicator ratio comprising at least two of the glucose data, the ketone data, and the lactate data;
determining, by the processor, an individualized metric by correlating the food intake information and the physical activity information to the global metric; and
recommending, by the processor, a behavior modification based on the individualized metric.

14. The method of claim 13, wherein:
the glucose data comprises a glucose variability (GV), a glucose load (GL), and a post-prandial peak (PPP);
the ketone data comprises a ketone load (KL) and a ketone variability (KV); and
the lactate data comprises a lactate load (LL) and lactate variability (LV).

15. The method of claim 14, wherein the indicator ratio is a GK Ratio of the glucose data and the ketone data, the GK Ratio being chosen from:
GK Ratio 1=GL/KL,
GK Ratio 2=GV/KV,
GK Mixed Ratio 1=GL/KV, and
GK Mixed Ratio 2=GV/KL.

16. The method of claim 14 wherein the indicator ratio is an LG Ratio of the lactate data and the glucose data, the LG Ratio being chosen from:
LG Ratio 1=GL/LL,
LG Ratio 2=GV/LV,
LG Mixed Ratio 1=GL/LV, and
LG Mixed Ratio 2=GV/LL.

17. The method of claim 14, wherein the indicator ratio is an LK Ratio of the lactate data and the ketone data, the LK Ratio being chosen from:
LK Ratio 1=LL/KL,
LK Ratio 2=LV/KV,
LK Mixed Ratio 1=LL/KV, and
LK Mixed Ratio 2=LV/KL.

18. The method of claim 13, wherein:
the calculating comprises calculating a plurality of global metrics; and
the determining comprises which global metric of the plurality of global metrics to correlate the food intake information and the physical activity information to.

19. The method of claim 13, wherein the data is metabolic data is provided by a continuous metabolic monitoring device.

20. The method of claim 13, wherein the behavior modification comprises a food parameter or an exercise parameter.
